(19) United States
(12) Patent Application Publication
(10) Pub. No.: US 2004/0010010 A1
(43) Pub. Date: Jan. 15, 2004

(54) Melanocortin receptor ligands

(76) Inventors: Frank Hallock Ebetino, Cincinnati, OH (US); Xuewei Liu, San Diego, CA (US); Mark Gregory Solinsky, Cincinnati, OH (US); John August Wos, Maineville, OH (US); Rashid Naeem Mumin, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/410,784

(22) Filed: Apr. 9, 2003

Related U.S. Application Data
(60) Provisional application No. 60/376,727, filed on Apr. 30, 2002.

(51) Int. Cl. A61K 31/454; A61K 31/445; C 07D 4 1/02; C07D 211/06

(52) U.S. Cl. 514/317; 514/326; 546/207; 546/226

(57) ABSTRACT

The present invention relates to compounds which comprise a 4-substituted piperidine ring linked to a substituted or unsubstituted hydrocarbyl ring. The compounds, including all enatiomeric and diasteriomeric forms and pharmaceutically acceptable salts thereof, have the formula:

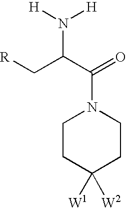

wherein preferably R is substituted aryl, W¹ is a carbocyclic unit, and W² is a heteroatom comprising unit.

### CROSS REFERENCE TO RELATED APPLICATIONS

This application claims priority under Title 35, United States Code 119(e) from Provisional Application Serial No. 60/376,727, filed Apr. 30, 2002.

### FIELD OF THE INVENTION

The present invention relates to melanocortin (MC) receptor ligands that have a 4-substituted piperidine ring, which provides for enhanced activity. These ligands preferably exhibit selectivity for the MC-3 and/or MC-4 receptors relative to the other melanocortin receptors (in particular the MC-1 receptor) and are suitable for use in pharmaceutical compositions and in treatment methods.

### BACKGROUND OF THE INVENTION

Melanocortin peptides (melanocortins) are natural peptide hormones in animals and man that bind to and stimulate MC receptors. Examples of melanocortins are α-MSH (melanocyte stimulating hormone), β-MSH, γ-MSH, ACTH (adrenocorticotropic hormone) and their peptide fragments. MSH is mainly known for its ability to regulate peripheral pigmentation, whereas ACTH is known to induce steroidoneogenesis. The melanocortin peptides also mediate a number of other physiological effects. They are reported to affect motivation, learning, memory, behavior, inflammation, body temperature, pain perception, blood pressure, heart rate, vascular tone, natriuresis, brain blood flow, nerve growth and repair, placental development, aldosterone synthesis and release, thyroxin release, spermatogenesis, ovarian weight, prolactin and FSH secretion, uterine bleeding in women, sebum and pheromone secretion, sexual activity, penile erection, blood glucose levels, intrauterine fetal growth, food motivated behavior, as well as other events related to parturition.

Both the MC-4 and MC-3 receptors have been localized to the hypothalamus, a region of the brain believed to be involved in the modulation of feeding behavior. Compounds showing selectivity for the MC-3/MC-4 receptors have been shown to alter food intake following intracerebroventricular and peripheral injection in rodents. Specifically, agonists have been shown to reduce feeding, while antagonists have been shown to increase feeding. The role of the MC-4 and MC-3 receptors have been defined in the control of body weight regulation in mammals. It is believed that the MC-3 receptor influences feed efficiency and the partitioning of fuel stores into fat, whereas the MC-4 receptor regulates food intake and possibly enery expenditure. Thus, these receptor subtypes appear to reduce body weight through distinct and complementary pathways. Therefore compounds that stimulate both the MC-3 and MC-4 receptors may have a greater weight loss effect than those that are selective for either the MC-3 or MC-4 receptor.

Body weight disorders such as obesity, anorexia and cachexia are widely recognized as significant public health issues and there is a need for compounds and pharmaceutical compositions which can treat these disorders. The Applicants have discovered a class of compounds that surprisingly have high affinity for the MC-4 and/or the MC-3 receptor subtypes, and that are typically selective for these MC receptors relative to the other melanocortin receptor subtypes, particularly the MC-1 subtype.

### SUMMARY OF THE INVENTION

It has now been surprisingly discovered that 4,4-disubstituted amino-piperidines are effective as melanocortin receptor ligands. These MC-4 agonists include all enatiomeric and diasteriomeric forms and pharmaceutically acceptable salts thereof, of compounds having the formula:

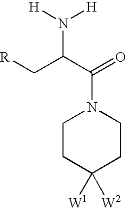

wherein R is a substituted or unsubstituted hydrocarbyl unit selected from the group consisting of:

a) non-aromatic carbocyclic rings;

b) aromatic carbocyclic rings;

c) non-aromatic heterocyclic rings;

d) aromatic heterocyclic rings;

W¹ is a pendant unit having the formula::

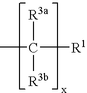

R¹ is selected from the group consisting of:

i) hydrogen;

ii) C₃-C₈ non-aromatic carbocyclic rings;

iii) C₆-C₁₄ aromatic carbocyclic rings;

iv) C₁-C₇ non-aromatic heterocyclic rings; and

v) C₃-C₁₃ aromatic heterocyclic rings;

R^{3a} and R^{3b} are each independently selected from the group consisting of

i) hydrogen;

ii) methyl; and

iii) R^{3a} and R^{3b} can be taken together to form a carbonyl unit;

the index x has the value from 0 to 10;

W² is a pendant unit having the formula:

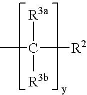

R² is selected from the group consisting of:

i) hydrogen;

ii) C₃-C₈ non-aromatic carbocyclic rings;

iii) C₆-C₁₄ aromatic carbocyclic rings;

iv) C₁-C₇ non-aromatic heterocyclic rings;

v) C₃-C₁₃ aromatic heterocyclic rings;

vi) —C(Y)R⁴;

vii) —C(Y)₂R⁴;

viii) —C(Y)N(R⁴)₂;

ix) —C(Y)NR⁴N(R⁴)₂;

x) —CN;

xi) —[C(R⁴)₂]C(R⁴)₂;

xii) —N(R⁴)₂;

xiii) —NR⁴CN;

xiv) —NR⁵C(Y)R⁴;

xv) —NR⁵C(Y)N(R⁴)₂;

xvi) —NHN(R⁴)₂;

xvii) —NHOR⁴;

xviii) —NO₂;

xix) —OR;

xx) and mixtures thereof;

Y is —O—, —S—, ═O, ═S, ═NR⁴, —R⁴, and mixtures thereof; R⁴ is hydrogen, C₁-C₄alkyl, —OH, and mixtures thereof; R⁵ is hydrogen, halogen, and mixtures thereof; M is hydrogen or a salt forming cation;

R^{3a} and R^{3b} are the same as above;

the index y has the value from 0 to 10.

The present invention further relates to pharmaceutical compositions comprising:

A) an effective amount of one or more melanocortin receptor ligands according to the present invention; and

B) one or more pharmaceutically acceptable excipients.

The present invention also relates to a method for controlling weight gain in a human or higher mammal, said method comprising the step of administering to said human or higher mammal an effective amount of one or more melanocortin receptor ligands according to the present invention.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference.

### DETAILED DESCRIPTION OF THE INVENTION

The present invention relates to melanocortin (MC) receptor ligands. The melanocortin (MC) class of peptides mediates a wide range of physiological effects. Synthetic peptides and peptide mimetics, which modulate the interaction of natural MC ligands have varying degrees of selectivity and binding. The present invention is directed to ligands that are selective for the MC4 receptor, or that are selective for both the MC4 and MC3 receptor while minimizing the interaction at the MC1, MC2, and MC5 receptors.

It has now been surprisingly discovered that 4,4-di-substituted amino-piperidines as described herein, are effective as melanocortin receptor ligands, especially as MC4 receptor ligands. The compounds of the present invention comprise a 4-piperidine ring position substitution which is a hydrocarbyl ring. In addition, the compounds of the present invention comprise a free amino group as defined by the formula below.

For the purposes of the present invention the term “hydrocarbyl” is defined herein as any organic unit or moiety which is comprised of carbon atoms and hydrogen atoms. Included within the term hydrocarbyl are the heterocycles which are described herein below. Examples of various unsubstituted non-heterocyclic hydrocarbyl units include pentyl, 3-ethyloctanyl, 1,3-dimethylphenyl, cyclohexyl, cis-3-hexyl, 7,7-dimethylbicyclo[2.2.1]-heptan-1-yl, and naphth-2-yl.

Included within the definition of “hydrocarbyl” are the aromatic (aryl) and non-aromatic carbocyclic rings, non-limiting examples of which include cyclopropyl, cyclobutanyl, cyclopentanyl, cyclohexane, cyclohexenyl, cycloheptanyl, bicyclo-[0.1.1]-butanyl, bicyclo-[0.1.2]-pentanyl, bicyclo-[0.1.3]-hexanyl (thujanyl), bicyclo-[0.2.2]-hexanyl, bicyclo-[0.1.4]-heptanyl (caranyl), bicyclo-[2.2.1]-heptanyl (norboranyl), bicyclo-[0.2.4]-octanyl (caryophyllenyl), spiropentanyl, diclyclopentanespiranyl, decalinyl, phenyl, benzyl, naphthyl, indenyl, 2H-indenyl, azulenyl, phenanthryl, anthryl, fluorenyl, acenaphthylenyl, 1,2,3,4-tetrahydronaphthalenyl, and the like.

The term “heterocycle” includes both aromatic (heteroaryl) and non-aromatic heterocyclic rings non-limiting examples of which include: pyrrolyl, 2H-pyrrolyl, 3H-pyrrolyl, pyrazolyl, 2H-imidazolyl, 1,2,3-triazolyl, 1,2,4-triazolyl, isoxazolyl, oxazoyl, 1,2,4-oxadiazolyl, 2H-pyranyl, 4H-pyranyl, 2H-pyran-2-one-yl, pyridinyl, pyridazinyl, pyrimidinyl, pyrazinyl, piperazinyl, s-triazinyl, 4H-1,2-oxazinyl, 2H-1,3-oxazinyl, 1,4-oxazinyl, morpholinyl, azepinyl, oxepinyl, 4H-1,2-diazepinyl, indenyl 2H-indenyl, benzofuranyl, isobenzofuranyl, indolyl, 3H-indolyl, 1H-indolyl, benzoxazolyl, 2H-1-benzopyranyl, quinolinyl, isoquinolinyl, quinazolinyl, 2H-1,4-benzoxazinyl, pyrrolidinyl, pyrrolinyl, quinoxalinyl, furanyl, thiophenyl, benzimidazolyl, and the like each of which can be substituted or unsubstituted.

The terms “arylene” and “heteroarylene” relate to aryl and heteroaryl units which can serve as part of a linking group, for example, units having the formula:

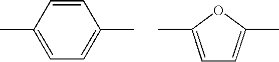

which represent an arylene and heteroarylene unit respectively.

The term “substituted” is used throughout the specification. The term “substituted” is defined herein as “encompassing moieties or units which can replace a hydrogen atom, two hydrogen atoms, or three hydrogen atoms of a hydrocarbyl moiety. Also substituted can include replacement of hydrogen atoms on two adjacent carbons to form a new moiety or unit.” For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. Three hydrogen replacement includes cyano, and the like. An epoxide unit is an example of a substituted unit which requires replacement of a hydrogen atom on adjacent carbons. The term substituted is used throughout the present specification to indicate that a hydrocarbyl moiety, inter alia, aromatic ring, alkyl chain, can have one or more of the hydrogen atoms replaced by a substituent. When a moiety is described as “substituted” any number of the hydrogen atoms may be replaced. For example, 4-hydroxyphenyl is a “substituted aromatic carbocyclic ring”, (N,N-dimethyl-5-amino)octanyl is a “substituted C₈ alkyl unit, 3-guanidinopropyl is a “substituted C₃ alkyl unit,” and 2-carboxypyridinyl is a “substituted heteroaryl unit.” The following are non-limiting examples of units which can serve as a replacement for hydrogen atoms when a hydrocarbyl unit is described as “substituted.”

i) —[C(R⁴)₂]ₚ(CH═CH)_{q}R⁴; wherein p is from 0 to 12; q is from 0 to 12;

ii) —C(X)R⁴;

iii) —C(X)₂R⁴;

iv) —C(X)CH═CH₂;

v) —C(X)N(R ⁴)₂;

vi) —C(X)NR⁴N(R⁴)₂;

vii) —CN;

viii) —CNO;

ix) —CF₃, —CCl₃, —CBr₃;

x) —N(R⁴)₂;

xi) —NR⁴CN;

xii) —NR⁴C(X)R⁴;

xiii) —NR⁴C(X)N(R⁴)₂;

xiv) —NHN(R⁴)₂;

xv) —NHOR⁴;

xvi) —NCS;

xvii) —NO₂;

xviii) —OR;

xix) —OCN;

xx) —OCF₃, —OCCl₃, —OCBr₃;

xxi) —F, —Cl, —Br, —I, and mixtures thereof;

xxii) —SCN;

xxiii) —SO₃M;

xxiv) —OSO₃M;

xxv) —SO₂N(R⁴)₂;

xxvi) —SO₂R⁴;

xxvii) —[C(R⁴)₂]ₙP(O)(OR⁴)R⁴;

xxviii) —[C(R⁴)₂]ₙP(O)(OR⁴)₂;

xxix) and mixtures thereof;

wherein R⁴ is hydrogen, C₁-C₄ linear, branched, or cyclic alkyl, halogen, —OH, —NO₂, —CN, and mixtures therof; M is hydrogen, or a salt forming cation; X is defined herein below. Suitable salt forming cations include, sodium, lithium, potassium, calcium, magnesium, ammonium, and the like. Non-limiting examples of an alkylenearyl unit include benzyl, 2-phenylethyl, 3-phenylpropyl, 2-phenylpropyl.

The compounds of the present invention include all enatiomeric and diasteriomeric forms and pharmaceutically acceptable salts of compounds having the core scaffold represented by the formula:

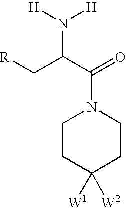

wherein R is a substituted or unsubstituted hydrocarbyl unit selected from the group consisting of:

a) non-aromatic carbocyclic rings;

b) aromatic carbocyclic rings;

c) non-aromatic heterocyclic rings;

d) aromatic heterocyclic rings;

A first aspect of R units relates to substituted and non-substituted aryl units wherein R units are substituted or unsubstituted phenyl, benzyl, naphthyl, and naphthalen-2-ylmethyl.

A first iteration of this aspect encompasses R units which are selected from the group consisting of phenyl, 4-fluorophenyl, 4-chlorophenyl, 4-hydroxyphenyl, and 4-methylphenyl. An example of this aspect which is particularly effective in enhancing MC-4 activity is 4-chlorophenyl, especially when combined with W¹ units comprising a carbocyclic ring, for example, cyclohexyl.

A second iteration of this aspect encompasses R units which are selected from the group consisting of 1-naphthyl, 2-naphthyl, naphthalen-1-ylmethyl, naphthalen-2-ylmethyl, and 1-hydroxynaphthalen-2-ylmethyl.

A second aspect of R units relates to substituted and non-substituted heteroaryl units wherein R units comprise substituted or unsubstituted quinolinyl, isoquinolinyl, tetrahydroquinolinyl, and tetrahydroisoquinolinyl.

A first iteration of this aspect encompasses R units which are 1,2,3,4-tetrahydro-isoquinolinyl and 1,2,3,4-tetrahydroquinolinyl.

A second iteration of this aspect encompasses R units which are 6-hydroxy-1,2,3,4-tetrahydroisoquinolinyl and 6-hydroxy-1,2,3,4-tetrahydroquinolinyl.

Another aspect of R relates to phenyl rings comprising a C₁-C₄ alkyl unit, non-limiting examples of which include 4-methylphenyl, 2,4-dimethylphenyl, as well as mixed alkyl rings, inter alia, 2-methyl-4-isopropyl.

A yet further aspect of R relates to substituted or unsubstituted heteroaryl rings selected from the group consisting of thiophenyl, furanyl, oxazolyl, thiazolyl, pyrrolyl, and pyridinyl.

W¹ is a pendant unit having the formula:

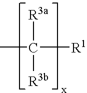

wherein R¹ is selected from the group consisting of:

i) hydrogen;

ii) C₃-C₈ non-aromatic carbocyclic rings;

iii) C₆-C₁₄ aromatic carbocyclic rings;

iv) C₁-C₇ non-aromatic heterocyclic rings; and

v) C₃-C₁₃ aromatic heterocyclic rings;

R^{3a} and R^{3b} are each independently selected from the group consisting of

i) hydrogen;

ii) methyl; and

iii) R^{3a} and R^{3b} can be taken together to form a carbonyl unit;

the index x has the value from 0 to 10.

The first aspect of W¹ relates units having the formula: having the formula:

—R¹

wherein the index x is 0. The first embodiment of this aspect relates to R¹ units which are substituted and unsubstituted carbocyclic rings selected from the group consisting of cyclopropyl, cyclopentyl, cyclohexyl, 2-methylenecyclopentyl, and cycloheptyl.

A second embodiment of this aspect relates to R¹ units which are aromatic or non-aromatic heterocyclic rings selected from the group consisting of thiophen-2-yl, piperidin-4-yl, pyridin-2-yl, and morpholin-4-yl.

The second aspect of W¹ relates to units having the formula:

—CH₂—R¹

wherein the index x is 1. The first embodiment of this aspect relates to R¹ units which are substituted and unsubstituted carbocyclic rings selected from the group consisting of cyclopropyl, cyclopentyl, cyclohexyl, 2-methylenecyclopentyl, and cycloheptyl.

A second embodiment of this aspect relates to R¹ units which are aromatic or non-aromatic heterocyclic rings selected from the group consisting of thiophen-2-yl, piperidin-4-yl, pyridin-2-yl, and morpholin-4-yl.

W² is a pendant unit having the formula:

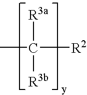

wherein R² is selected from the group consisting of:

i) hydrogen;

ii) C₃-C₈ non-aromatic carbocyclic rings;

iii) C₆-C₁₄ aromatic carbocyclic rings;

iv) C₁-C₇ non-aromatic heterocyclic rings;

v) C₃-C₁₃ aromatic heterocyclic rings;

vi) —C(Y)R⁴;

vii) —C(Y)₂R;

viii) —C(Y)N(R⁴)₂;

ix) —C(Y)NR⁴N(R⁴)₂;

x) —CN;

xi) —[C(R⁴)₂]C(R⁴)₂;

xii) —N(R⁴)₂;

xiii) —NR⁴CN;

xiv) —NR⁵C(Y)R⁴;

xv) —NR⁵C(Y)N(R⁴)₂;

xvi) —NHN(R⁴)₂;

xvii) —NHOR⁴;

xviii) —NO₂;

xix) —OR⁴;

xx) and mixtures thereof;

Y is —O—, —S—, ═O, ═S, ═NR⁴, —R⁴, and mixtures thereof; R⁴ is hydrogen, C₁-C₄ linear, branched, or cyclic alkyl, halogen, —OH, —NO₂, —CN, and mixtures thereof; R⁵ is hydrogen, halogen, and mixtures thereof; M is hydrogen or a salt forming cation.

R^{3a} and R^{3b} are the same as defined herein above.

The index y has the value from 0 to 10.

One aspect of the present invention relates to W² units which are short chain alkyl or alkenyl (lower hydrocarbyl) esters having the formula:

—C(O)OR⁴;

non-limiting examples of which are —C(O)OCH₃; —C(O)OCH₂CH₃; —C(O)OCH₂CH₂CH₃; —C(O)OCH₂CH₂CH₂CH₃; —C(O)OCH(CH₃)₂; —C(O)OCH₂CH(CH₃)₂; —C(O)OCH₂CH═CHCH₃; —C(O)OCH₂CH₂CH(CH₃)₂; —C(O)OCH₂C(CH₃)₃; and the like.

Another aspect of the present invention relates to W² units which are short chain substituted or non-substituted amides having the formula:

—C(O)NHR⁴ or —NHC(O)R⁴

non-limiting examples of which are —C(O)NHCH₃; —C(O)NHCH₂CH₃; —C(O)NHCH(CH₃)₂; —C(O)NHCH₂CH₂CH₃; —C(O)NHCH₂CH₂CH₂CH₃; —C(O)NHCH₂CH(CH₃)₂; —C(O)NH₂; —C(O)NHCH₂CH═CHCH₃; —C(O)NHCH₂CH₂CH(CH₃)₂; —C(O)NHCH₂C(CH₃)₃; —C(O)NHCH₂CH₂SCH₃; —C(O)NHCH₂CH₂OH; —NHC(O)CH₃; —NHC(O)CH₂CH₃; —NHC(O)—CH₂CH₂CH₃; and the like.

Another aspect of the present invention as it relates to W² units encompasses units having the formula:

(CH₂)_{y}—R²

wherein the index y is from 1 to 3.

A first iteration of this aspect relates to R² units which are heterocycles selected from the group consisting of:

i) thiazolyl, 2-methylthiazolyl, 4-mentylthiazolyl, 5-methylthiazolyl having the formula:

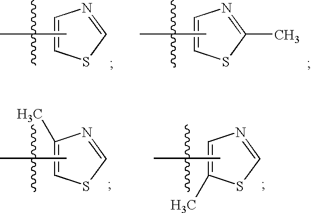

ii) 1,3,4-thiadiazolyl, 2-methyl-1,3,4-thiadiazolyl having the formula:

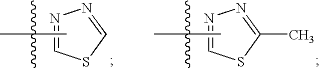

iii) 1,2,5-thiadiazolyl, 3-methyl-1,2,5-thiadiazolyl having the formula:

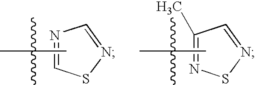

iv) oxazolyl, 2-methyloxazolyl, 4-methyloxazolyl, 5-methyloxazolyl having the formula:

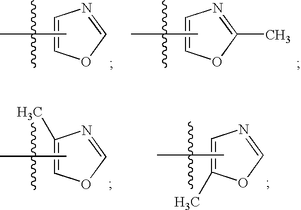

v) imidazolyl, 2-methylimidazolyl, 5-methylimidazolyl having the formula:

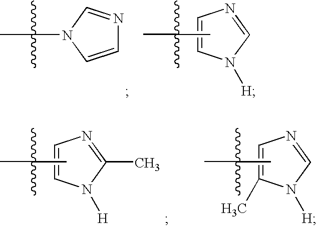

vi) 5-methyl-1,2,4-oxadiazolyl, 2-methyl- 1,3,4-oxadiazolyl, 5-amino- 1,2,4-oxadiazolyl, having the formula:

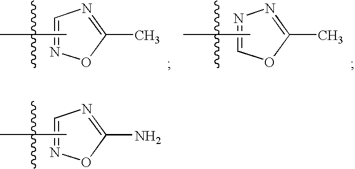

vii) 1,2-dihydro[1,2,4]triazol-3-one-1-yl, 2-methyl- 1,2-dihydro[1,2,4]triazol-3-one-5-yl, having the formula:

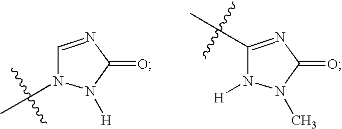

viii) oxazolidin-2-one-3-yl; 4,4-dimethyloxazolidin-2-one-3-yl; imidazolidin-2-one-1-yl; 1-methylimidazolidin-2-one-1-yl, having the formula:

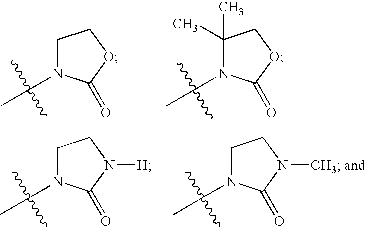

ix) 2-methyl-1,3,4-oxadiazolyl, 2-amino-1,3,4-oxadiazolyl, 2-(N,N-dimethylamino)-1,3,4-oxadiazolyl, having the formula:

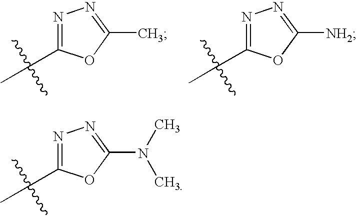

A second iteration of this aspect relates to R² units which are selected from the group consisting of:

i) triazoles having the formula:

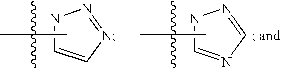

ii) tetrazole having the formula:

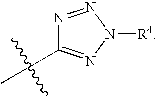

Non-limiting examples of scaffolds comprising the heterocycles of this aspect include:

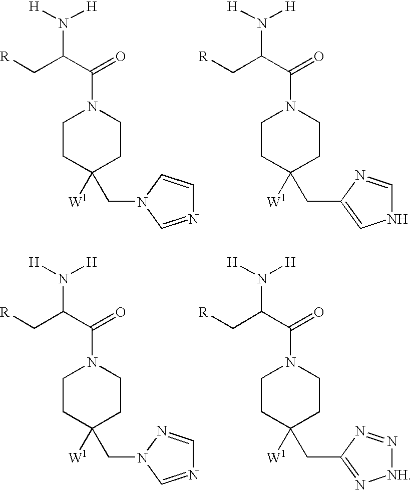

A further aspect of the present invention relates to W² units having the formula:

—(CH₂)_{y}—R²

the index y is 1, 2, or 3 and R is selected from the group consisting of:

a) —C(O)N(R⁴)₂;

b) —C(O)NR⁴N(R⁴)₂;

c) —NR⁴C(O)N(R⁴)₂; and

d) —NR⁴C(═NR⁴)N(R⁴)₂;

R⁴ is hydrogen, methyl, and mixtures thereof; R⁴ is hydrogen, methyl, —NO₂, —CN, and mixtures thereof.

Non-limiting examples of W² units comprising this aspect have the formula:

a) —(CH₂)_{y}NHC(O)NH₂;

b) —(CH₂)_{y}NHC(═NH)NH₂;

c) —(CH₂)_{y}NHC(═NCH₃)NHCN;

d) —(CH₂)_{y}NHC(═NNO₂)NHCN;

e) —(CH₂)_{y}NHC(═NCH₃)NHNO₂;

f) —(CH₂)_{y}NHC(═NCN)NHNO₂; and

g) —(CH₂)_{y}NHC(═NCN)NH₂;

wherein y is 1, 2, or 3. A first iteration includes W² units wherein y is equal to 3 and R² has the formula:

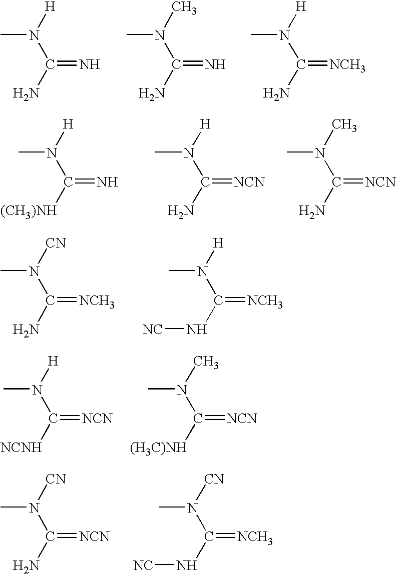

A further aspect of R² includes substituted or unsubstituted 6-member ring heterocycles selected from the group consisting of pyranyl, 1,4-dioxanyl, morpholinyl, pyridinyl, pyridazinyl, pyrimidinyl, pyrazinyl, piperidinyl, piperazinyl, triazinyl, 1,4-dithianyl, and thiomorpholinyl.

As further described herein below, one category of melanocortin receptor ligands according to the present invention relates to compounds selected from the group consisting of:

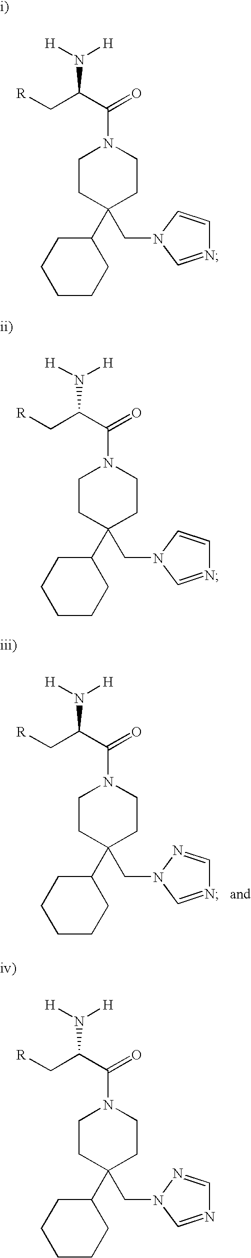

wherein R includes 4-chlorophenyl, 4-fluoropheny, and phenyl. Although all enantiomers and diastereomers are include within the structures depicted in the present invention, the following convention applies throughout the present specification.

The chemical name:

2-Amino-3-(4-chlorophenyl)-1-(4-cyclohexyl-4-[1,2,4]triazol-1-yl methyl-piperidin-1-yl) propan-1-one;

stands equally well for:

2-(R)-Amino-3-(4-chlorophenyl)-1-(4-cyclohexyl-4-[1,2,4]triazol -1-ylmethyl-piperidin-1-yl) propan-1-one;

and for:

2-(S)-Amino-3-(4-chlorophenyl)-1-(4-cyclohexyl-4-[1,2,4]triazol-1-ylmethyl-piperidin-1-yl) propan-1-one;

as well as the pharmaceutically acceptable salts thereof, inter alia, trifluoroacetate.

A further example of this convention relates to the analogs having the chemical name:

2-Amino-3-(4-chlorophenyl)-1-(4-cyclohexyl-4-imidazol-1-ylmethyl-piperidin-1-yl) propan-1-one;

stands equally well for:

2-(R)-Amino-3-(4-chlorophenyl)-1-(4-cyclohexyl-4-imidazol-1-ylmethyl-piperidin-1-yl) propan-1-one;

and for:

2-(S)-Amino-3-(4-chlorophenyl)-1-(4-cyclohexyl-4-imidazol-1-ylmethyl-piperidin-1-yl) propan-1-one.

In addition, and chiral centers in the following examples can have the reversed configuration and the procedures and reactions will act equally well, for example, R and be S and vice versa.

### Preparation of Melanocortin Receptor Ligands

The melanocortin receptor ligands of the present invention have the formula:

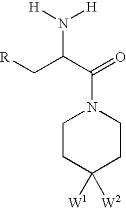

and said ligands can be prepared by the coupling of a lower portion comprising a 4,4-disubstituted piperidine, or protected variation thereof, with an upper portion which comprises the free amino terminus of the molecule, typically as a nitrogen protected precursor. This strategy can be summarized by the scheme below:

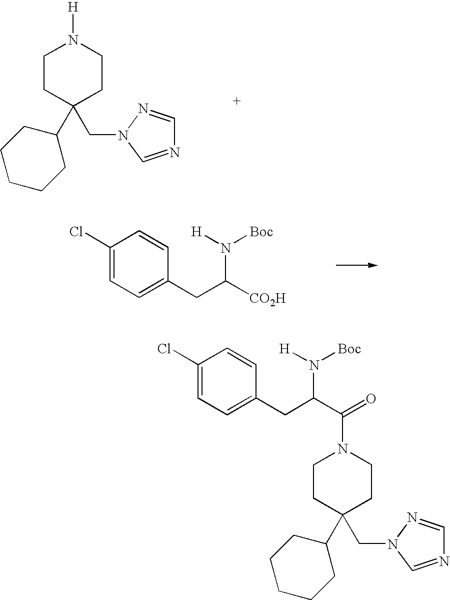

wherein 4-cyclohexyl-4-[1,2,4]triazolylmethylpiperidine and N-Boc-(4-chlorophenyl)alanine are condensed under routine conditions. Removal of the N-protecting group on the amino-comprising upper portion affords the final melanocortin receptor ligand.

The 4,4-disubstituted piperidine portion of the final molecule can be prepared prior to the condensation step. The 4-cyclohexylpiperidine scaffold is used in the examples which follow to illustrate convenient procedures for preparing the analogs of the present invention. These examples illustrate how intermediates comprising various forms of the W¹ unit can be integrated into a simple convergent synthetic pathway.

One precursor useful in preparing melanocortin receptor ligands relates to the hydroxy adduct: 4-cyclohexyl-4-hydroxymethyl-piperidine-1-carboxylic acid tert-butyl ester via the scheme outlined below.

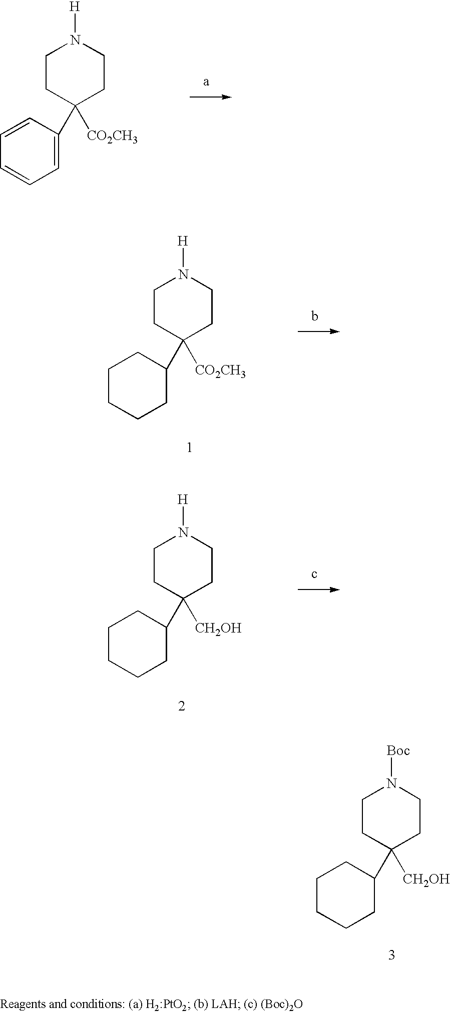

Preparation of 4-cyclohexylpiperidine-4-carboxylic acid ethyl ester (1): To a solution of 4-phenylpiperidine-4-carboxylic acid ethyl ester (56 g, 248 mmol) in EtOH (700 mL) is added platinum (IV) oxide (10.2 g, 45 mmol) and concentrated hydrochloric acid. The Flask is purged with nitrogen and shaken on a Parr hydrogenation apparatus at 40 psig for 18 hours. The flask is removed and additional PtO₂ (2 g, 8.8 mmol) is added and hydrogenation is continued at 40 psig an additional 6 hours. The reaction solution is filtered to remove the catalyst and the filtrated is concentrated in vacuo to afford a residue which is partitioned between saturated NaHCO₃ and methylene chloride. The organic phase is removed and the aqueous phase washed several times with methylene chloride. The organic layers are combined, dried and concentrated under in vacuo to afford the desired product in nearly quantitative yield as a waxy solid. ¹H NMR (300 MHz, CDCl₃) δ0.90-1.45 (m, 6H),1.25-1.32 (t, 3H), 1.55-1.85 (m, 7H), 2.15-2.28 (m, 2H), 2.98-2.80 (m, 2H), 3.18-3.27 (m, 2H), 4.10-4.25 (m, 2H), 7.10 (broad s, 1H); MS (ESI) m/z 240, (M+H⁺).

Preparation of (4-cyclohexylpiperidin-4-yl)-methanol (2): To a cooled (−5° C.) solution of lithium aluminum hydride (900 mL, 0.90 moles, 1.0M solution in THF) is added tetrahydrofuran (2000 mL) and 4-cyclohexyl-piperidine-4-carboxylic acid ethyl ester, 1, (59.5 g, 249 mmol). The resulting solution is stirred at between −5° C. and +3° C. for 1 hour and then allowed to warmed to room temperature and stir an additional sixty-six hours. The reaction is then re-cooled to 0° C. and carefully quenched with saturated ammonium chloride (100 mL). The reaction mixture is stirred for 10 minutes and then 87:10:3 ethyl acetate:methanol:triethylamine (500 mL) is added. The suspension is then stirred at room temperature for 20 minutes and filtered through a pad of Celite. The solids are re-suspended in 1:1 THF:EtOAc (2000 mL), stirred at room temperature for 1 hour and the suspension was again filtered through a pad of Celite. The filtrates are combined and concentrated in vacuo to afford 53.6 g of a mixture of the desired compound and 4-cyclohexyl-piperidine-4-carbaldehyde. The crude mixture is used directly in without further purification.

Preparation of 4-cyclohexyl-4-hydroxymethylpiperidine-1-carboxylic acid tert-butyl ester (3): Di-tert-butyl dicarbonate (79 g, 362 mmol) is added to a stirred solution of (4-cyclohexyl-piperidin-4-yl)-methanol, 2, (53.6 g) and triethylamine (180 mL) in MeOH (1600 mL) at 0° C. The resulting solution is allowed to warm to room temperature and is stirred an additional 4 hours. The solution concentrated in vacuo and purified via chromatography eluting with EtOAc/hexane 3:2, to afford 35.8 g (48% yield) of the desired product as a white solid. ¹H NMR (300 MHz, CDCl₃) δ1.00-1.32 (m, 5H), 1.35-1.60 (m, 14H), 1.65-1.88 (m, 5H), 3.15-3.30 (m, 2H), 3.48-3.65 (m, 2H), 3.63 (s, 2H); MS (ESI) m/z 298, (M+H⁺).

From intermediate compound 3, a series of other precursors useful in preparing melanocortin receptor ligands can be obtained. The mesylate 4 can be used to introduce a variety of 4-position-substituted piperidine, for example, triazole 5:

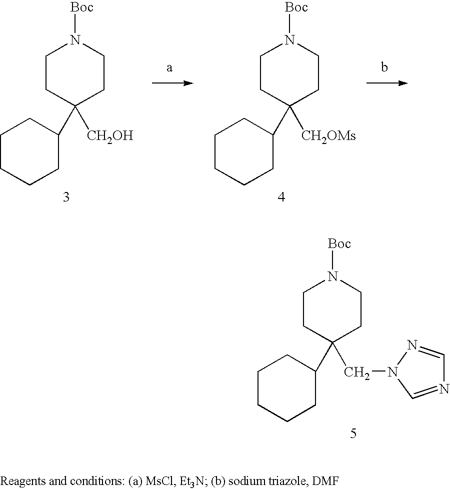

or azide 6 which can be used to introduce a variety of functional groups as further described herein below.

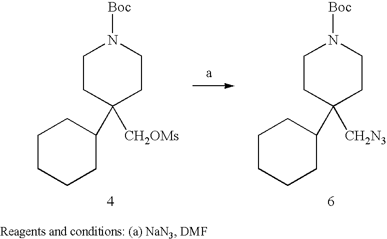

Preparation of 4-cyclohexyl-4-methanesulfonyloxymethylpiperidine-1-carboxylic acid tert-butyl ester (4): Methane sulfonyl chloride (1.8 mL, 23.0 mmol) is added to a stirred solution of 4-cyclohexyl-4-hydroxymethylpiperidine-1-carboxylic acid tert-butyl ester, 3, (3.42 g, 11.48 mmol) and triethylamine (4.8 mL, 2.8 mmol) in dichloromethane (30 mL) at 0° C. The reaction mixture is then allowed to warm to room temperature and stir for 1 hour. The reaction is quenched with a saturated solution of NaHCO₃ and the resulting mixture is extracted twice with dichloromethane (50 mL). The organic layers are combined, dried, filtered and concentrated in vacuo to yield the desired product in quantitative yield. The material is used for the next step without need for purification.

Preparation of 4-cyclohexyl-4-[1,2,4]triazol-1-ylmethyl-piperidine-1-carboxylic acid tert-butyl ester (5): To a solution of 4-cyclohexyl-4-methansulfonyloxymethyl-piperidine-1-carboxylic acid tert-butyl ester (39 g, 103.8 mmol) in N,N-dimethylformamide (200 mL) is added sodium triazole (38 g, 415.2 mmol). The resulting solution is heated to 100° C. for 24 hours then cooled to room temperature. The solvent is removed under reduce pressure and the crude product purified over silica (80:20 EtOAc:hexane) to afford 28.7 g (79.7% yield) of the desired compound as a colorless solid. ¹H NMR (CD₃OD) δ0.95-1.90 (m, 15H), 1.46 (s, 9H), 3.45-3.55 (m, 4H), 4.34 (s, 2H), 7.99 (s, 1H), 8.48 (s, 1H). MS (ESI) m/z 349, (M+H⁺), 371(M+Na⁺)

Preparation of 4-cyclohexyl-4-azidomethylpiperidine-1-carboxylic acid tert-butyl ester (6): To a solution of 4-cyclohexyl-4-methanesulfonyloxymethyl-piperidine-1-carboxylic acid tert-butyl ester, 4, (2.42 g, 6.73 mmol) in DMF (25 mL) is added sodium azide (1.32 g, 20.2 mmol) and the mixture is heated and stirred at 100° C. over night. The reaction is cooled and then quenched with water. The resulting solution is extracted with EtOAc (30 mL), dried, filtered and concentrated in vacuo to afford the crude product as a brown oil which is purified via chromatography on silica gel eluting with hexane/EtOAc 3:1 to afford the desired product in 76% yield (1.91 g) as a colorless oil.

The intermediate aldehyde 7 can be used to prepare various W² units.

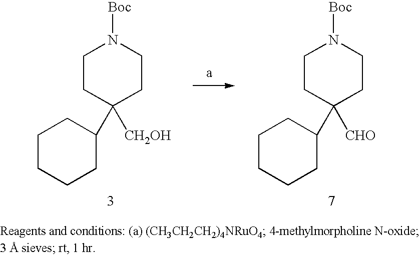

Preparation of 4-cyclohexyl-4-formyl-piperidine-1-carboxylic acid tert-butyl ester (7): To a mixture of 4-cyclohexyl-4-hydroxymethyl-piperidine-1-carboxylic acid tert-butyl ester, 3, (1.0 g, 3.36 mmol), 4-methylmorpholine N-oxide (0.54 g, 4.64 mmol), and molecular sieves (0.5 g) in methylene chloride (20 mL) under argon atmosphere is added tetrapropylammonium perruthenate (35.5 mg) at room temperature. The mixture is stirred for 30 min to 1 hour after which the solution is filtered through a pad of silica and the solvent removed in vacuo to afford the desired product as a colorless oil, which is used without further purification. MS (ESI) m/z 318, (M+Na⁺).

The following are non-limiting examples of functional groups and functional group precursors which can be prepared from aldehyde 7.

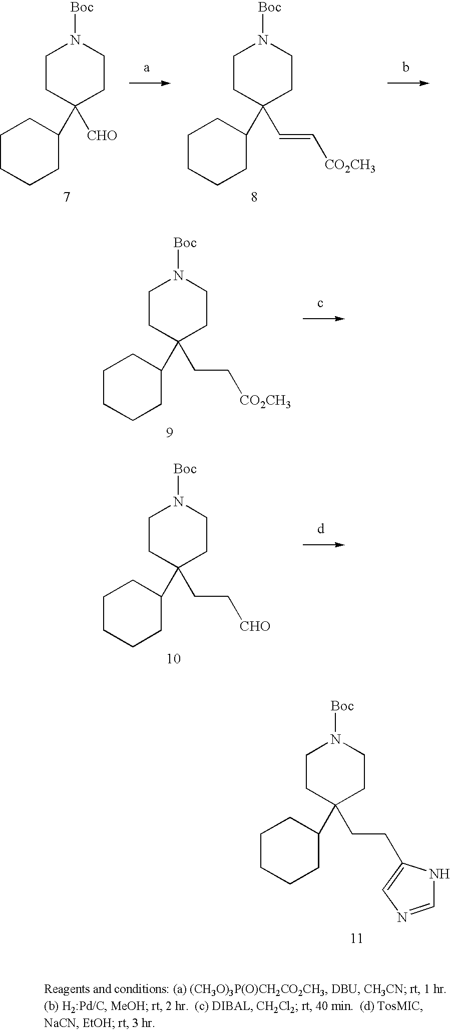

Preparation of 4-cyclohexyl-4-(2-methoxycarbonyl-vinyl)-piperidine-1-carboxylic acid tert-butyl ester (8): To a solution of trimethyl phosphonoacetate (1.41 ml, 8.72 mmole), lithium chloride (477 mg, 11.3 mmole), and 1,8-diazabicyclo[4.3.0]non-7-ene (DBU) (1.55 ml, 11.3 mmole) in anhydrous acetonitrile (25 ml) is added 4-cyclohexyl-4-formyl-piperidine-1-carboxylic acid tert-butyl ester, 7, (2.58 mg, 8.72 mmole) under argon at room temperature. The mixture is stirred for one hour and the solvent then removed under reduced pressure. The crude product is purified over silica (methylene chloride:methanol=15:1, R_{f}=0.78) to afford 2.64 g (86% yield) of the desired compound.

Preparation of 4-cyclohexyl-4-(2-methoxycarbonyl-ethyl)-piperidine-1-carboxylic acid tert-butyl ester (9): To a solution of 4-cyclohexyl-4-(2-methoxycarbonyl-vinyl)-piperidine-1-carboxylic acid tert-butyl ester, 8, (2.64 g, 7.5 mmole) in methanol (30 ml) is added 10% palladium on carbon (120 mg) under argon. The mixture is purged with hydrogen and then stirred for two hours under a hydrogen atmosphere at atmospheric pressure. The reaction mixture is filtered through a short pad of Celite and the filtrate concentrated under reduced pressure. The crude product is purified over silica to afford 2.57 g (97% yield) of the desired compound.

Preparation of 4-cyclohexyl-4-(3-oxo-propyl)-piperidine-1-carboxylic acid tert-butyl ester (10): To a cooled (−78° C.) solution of 4-cyclohexyl-4-(2-methoxycarbonyl-ethyl)-piperidine-1-carboxylic acid tert-butyl ester, 9, (1.0 g, 2.833 mmole) in 40 ml of anhydrous methylene chloride is added diisobutylaluminum hydride (5.75 ml, 1 M, 5.75 mmole). The reaction is stirred at room temperature for 40 min before it is quenched by adding methanol (3 ml) and water (20 ml). The reaction mixture is warmed to room temperature and the organic layer separated, dried over sodium sulfate, filtered and concentrated in vacuo to afford 915 mg (>99% yield) of the desired compound as a colorless oil.

Preparation of 4-cyclohexyl-4-[2-(3H-imidazol-4-yl)-ethyl]-piperidine-1-carboxylic acid tert-butyl ester (11): A solution of 4-cyclohexyl-4-(3-oxo-propyl)-piperidine-1-carboxylic acid tert-butyl ester, 10, (300 mg, 0.93) in ethanol (10 ml) is treated with tosylmethyl isocyanide (tosMIC) (176 mg, 0.93 mmole) and sodium cyanide (6 mg) at room temperature for three hours. The solvent is removed under reduced pressure and ammonia in methanol (2M, 10 ml) added. The mixture is stirred in a sealed tube overnight. The reaction mixture is then concentrated under reduced pressure and the residue taken up in chloroform, washed with aqueous sodium bicarbonate, brine, then dried with sodium sulfate and concentrated to a red oil. The residue is purified over silica (methylene chloride:methanol=15:1, R_{f}=0.58) to afford 141 mg (42% yield) of the desired product.

The compounds which comprise Category I of the melanocortin receptor ligands of the present invention are 4-cyclohexyl-4-[1,2,4]triazol-1-yl piperidines having the general scaffold:

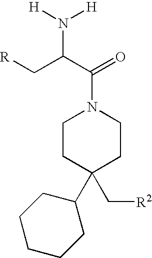

wherein R and R² are defined herein below in Table I.

TABLE I

| No. | R | R² |
|---|---|---|
| 1 | phenyl | —NH₂ |
| 2 | phenyl | imidazol-1-yl |
| 3 | phenyl | imidazol-2-yl |
| 4 | phenyl | imidazol-4-yl |
| 5 | phenyl | 1-methylimidazol-4-yl |
| 6 | phenyl | [1,2,4]triazol-1-yl |
| 7 | phenyl | —NHC(O)NHCH₃ |
| 8 | phenyl | —NHC(═NCN)NHCH₃ |
| 9 | phenyl | —NHC(═NCH₃)SCH₃ |
| 10 | phenyl | —NH(C═S)NHCH₃ |
| 11 | phenyl | (thiazol-2-yl)amino |
| 12 | phenyl | tetrazolyl |
| 13 | 4-fluorophenyl | —NH₂ |
| 14 | 4-fluorophenyl | imidazol-1-yl |
| 15 | 4-fluorophenyl | imidazol-2-yl |
| 16 | 4-fluorophenyl | imidazol-4-yl |
| 17 | 4-fluorophenyl | 1-methylimidazol-4-yl |
| 18 | 4-fluorophenyl | [1,2,4]triazol-1-yl |
| 19 | 4-fluorophenyl | —NHC(O)NHCH₃ |
| 20 | 4-fluorophenyl | —NHC(═NCN)NHCH₃ |
| 21 | 4-fluorophenyl | —NHC(═NCH₃)SCH₃ |
| 22 | 4-fluorophenyl | —NH(C═S)NHCH₃ |
| 23 | 4-fluorophenyl | (thiazole-2-yl)amino |
| 24 | 4-fluorophenyl | tetrazolyl |
| 25 | 4-chlorophenyl | —NH₂ |
| 26 | 4-chlorophenyl | imidazol-1-yl |
| 27 | 4-chlorophenyl | imidazol-2-yl |
| 28 | 4-chlorophenyl | imidazol-4-yl |
| 29 | 4-chlorophenyl | 1-methylimidazol-4-yl |
| 30 | 4-chlorophenyl | [1,2,4]triazol-1-yl |
| 31 | 4-chlorophenyl | —NHC(O)NHCH₃ |
| 32 | 4-chlorophenyl | —NHC(═NCN)NHCH₃ |
| 33 | 4-chlorophenyl | —NHC(═NCH₃)SCH₃ |
| 34 | 4-chlorophenyl | —NH(C═S)NHCH₃ |
| 35 | 4-chlorophenyl | (thiazole-2-yl)amino |
| 36 | 4-chlorophenyl | tetrazolyl |
| 37 | 4-hydroxyphenyl | —NH₂ |
| 38 | 4-hydroxyphenyl | imidazol-1-yl |
| 39 | 4-hydroxyphenyl | imidazol-2-yl |
| 40 | 4-hydroxyphenyl | imidazol-4-yl |
| 41 | 4-hydroxyphenyl | 1-methylimidazol-4-yl |
| 42 | 4-hydroxyphenyl | [1,2,4]triazol-1-yl |
| 43 | 4-hydroxyphenyl | —NHC(O)NHCH₃ |
| 44 | 4-hydroxyphenyl | —NHC(═NCN)NHCH₃ |
| 45 | 4-hydroxyphenyl | —NHC(═NCH₃)SCH₃ |
| 46 | 4-hydroxyphenyl | —NH(C═S)NHCH₃ |
| 47 | 4-hydroxyphenyl | (thiazole-2-yl)amino |
| 48 | 4-hydroxyphenyl | tetrazolyl |

The following is a scheme for preparing analogs encompassed by Category I of the melanocortin receptor ligands of the present invention.

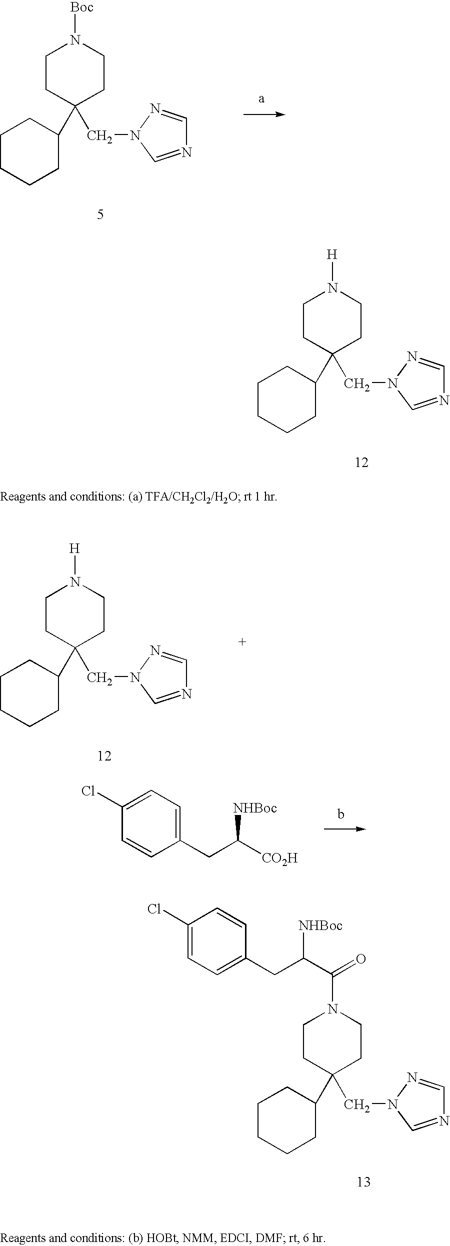

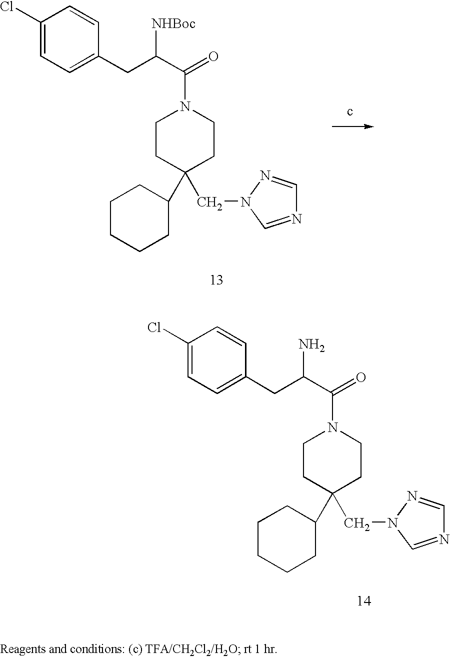

### EXAMPLE 1

### 2-Amino-3-(4-chlorophenyl)-1-(4-cyclohexyl-4-[1,2,4]triazol-1-ylmethyl-piperidin-1-yl)propan-1-one (14)

Preparation of 4-cyclohexyl-4-[1,2,4]triazol-1-ylmethylpiperidine (12): To a solution of trifluoroacetic acid/dichloromethane/water (1:1:0.1, 10 mL) is added to 4-cyclohexyl-4-[1,2,4]triazol-1-ylmethyl-piperidine-1-carboxylic acid tert-butyl ester, 5, (3.5 g, 10 mmol) is added to the residue obtained in the procedure herein above and the reaction mixture is allowed to stir for 30 to 60 minutes. The reaction solution is then concentrated in vacuo and partitioned between aqueous NaHCO₃ and EtOAc. The organic phase is concentrated in vacuo and the crude product purified by HPLC over silica gel to afford the desired product.

Preparation of [1-(4-chlorobenzyl)-2-(4-cyclohexyl-4-[1,2,4]triazol-1-ylmethyl-piperidin-1-yl)-2-oxo-ethyl]carbamic acid tert-butyl ester (13): To a solution of 4-cyclohexyl-4-[1,2,4]triazol-1-ylmethylpiperidine, 12, (2.16 g, 8.74 mmol), (R)-2-N-(tert-butoxy-carbonyl)-amino-3-(4-chloro)phenyl-propanoic acid [Boc-D-Ph(p-Cl)-OH] (2.65 g, 9.18 mmol), 1-hydroxy-benzotriazole (2.36 g, 17.5 mmol), N-methylmorpholine (35.0 mmol, 3.83 mL) in DMF (30 mL) is added in portions 1-(3-dimethylaminopropyl)-3-ethylcarbodiimide hydrochloride (2.16 g, 11.4 mmol). The reaction is allowed to stir for 6 hours after which it is quenched by adding aqueous NH₄Cl. The reaction mixture is extracted with EtOAc and the combined layers are dried, concentrated in vacuo, and the resulting crude product purified over silica gel to afford the desired product.

Preparation of 2-amino-3-(4-chlorophenyl)-1-(4-cyclohexyl-4-[1,2,4]triazol-1-ylmethyl-piperidin-1-yl)propan-1-one (14): A solution of trifluoroacetic acid/dichloromethane/water (1:1:0.1, 5 mL) is added to [1-(4-chlorobenzyl)-2-(4-cyclohexyl-4-[1,2,4]triazol-1-ylmethyl-piperidin-1-yl)-2-oxo-ethyl]carbamic acid tert-butyl ester, 13, (3.5 g, 6.65 mmol) and the reaction mixture is allowed to stir for 30 to 60 minutes. The reaction solution is then concentrated in vacuo and partitioned between aqueous NaHCO₃ and EtOAc. The organic phase is concentrated in vacuo and the crude product purified via HPLC over silica gel to afford the desired product.

The following scheme utilizes intermediate 6 for the preparation of other Category I analogs.

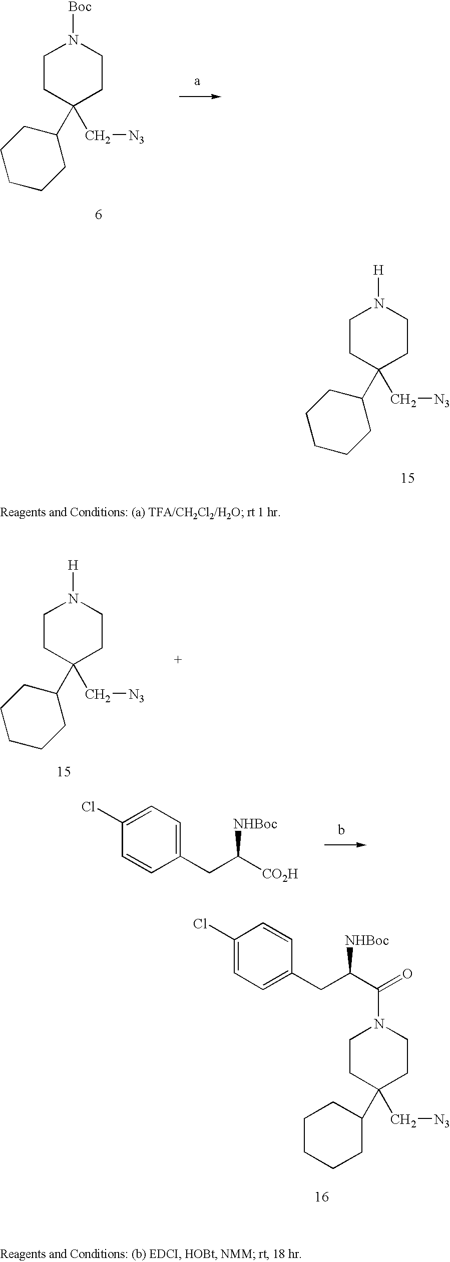

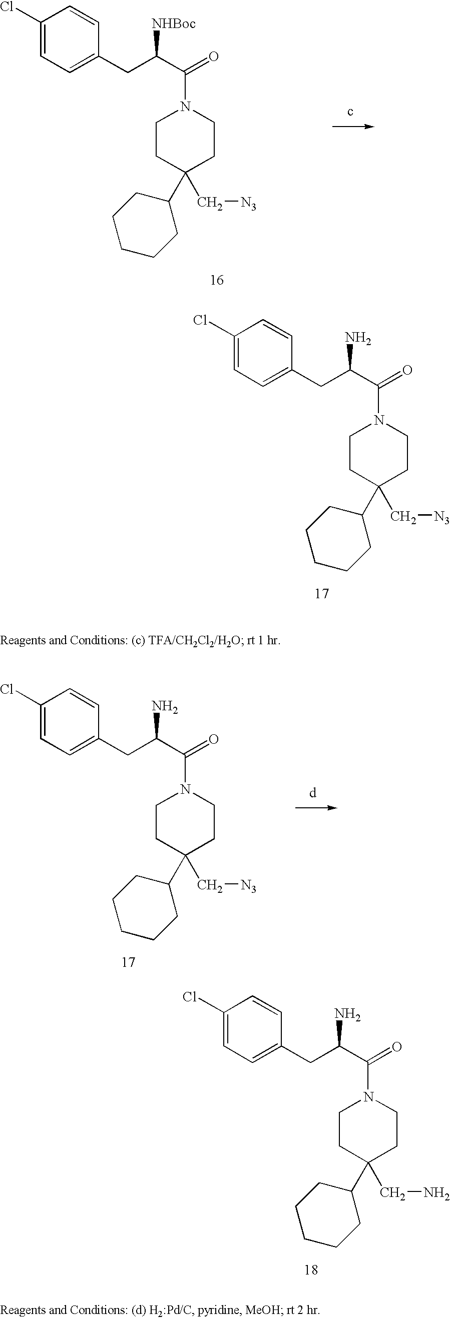

### EXAMPLE 2

### 2-(R)-Amino-1-(4-aminomethyl-4-cyclohexyl-piperidin-1-yl)-3-(4-chlorophenyl)-propan-1-one (18)

Preparation of 4-azidomethyl-4-cyclohexyl-piperidine (15): A ready-to-use solution of trifluoroacetic acid:methylene chloride:water (1:1:0.1, 20 ml) is added to 4-azidomethyl-4-cyclohexyl-piperidine-1-carboxylic acid tert-butyl ester, 6, (1.91 g, 5.92 mmole), and the reaction mixture stirred for 0.5-1.0 hour. The reaction is then concentrated under reduced pressure and partitioned between aqueous sodium bicarbonate and ethyl acetate. The organics are separated and the solvent removed under reduced pressure. The crude product is purified by preparative HPLC to afford 1.32 g (100% yield) of the desired product as the trifluoroacetic acid salt. MS (ESI) m/z 223, (M+H⁺)

Preparation of [2-(4-azidomethyl-4-cyclohexyl-piperidin-1-yl)-1-R-(4-chlorobenzyl)-2-oxo-ethyl]-carbamic acid tert-butyl ester (16): To a solution of the 4-azidomethyl-4-cyclohexyl-piperidine, 15, (1.95 g, 8.74 mmol), 2-(R)-tert-butoxycarbonylamino-3-(4-chloro-phenyl)-propionic acid (2.65 g, 9.18 mmol), 1-hydroxybenzotriazole (2.36 g, 17.5 mmol), 4-methyl-morpholine (35.0 mmole, 3.83 mL) in N,N-dimethylformamide (30 mL) is added 1-(3-dimethyl-aminopropyl)-3-ethylcarbodiimide (2.16 g, 11.4 mmol) and the reaction mixture is stirred overnight. Aqueous ammonium chloride solution is then added and the reaction extracted with ethyl acetate. The organic layer is dried over sodium sulfate, filtered and concentrated under reduced pressure. The crude product is purified by chromatography to afford 3.35 g (76% yield) of the title compound. MS (ESI) m/z 504, (M+H⁺)

Preparation of 2-(R)-amino-1-(4-azidomethyl-4-cyclohexyl-piperidin-1-yl)-3-(4-chlorophenyl)-propan-1-one (17): A ready-to-use solution of trifluoroacetic acid:methylene chloride:water (1:1:0.1, 15 ml) is added to [2-(4-azidomethyl-4-cyclohexyl-piperidin-1-yl)-1-R-(4-chlorobenzyl)-2-oxo-ethyl]-carbamic acid tert-butyl ester, 16, (3.35 g, 6.65 mmole), and the reaction mixture stirred for 0.5-1.0 hour. The mixture s concentrated under reduced pressure and partitioned between aqueous sodium bicarbonate and ethyl acetate. The organics are separated and the solvent removed under reduced pressure. The crude product is purified by preparative HPLC to afford 2.68 g (99% yield) of the desired product. MS (ESI) m/z 404, (M+H⁺)

Preparation of 2-(R)-amino-1-(4-aminomethyl-4-cyclohexyl-piperidin-1-yl)-3-(4-chloro-phenyl)-propan-1-one (18): To a solution of 2-(R)-amino-1-(4-azidomethyl-4-cyclohexyl-piperidin-1-yl)-3-(4-chloro-phenyl)-propan-1-one, 17, (2.68, 6.7 mmole) and pyridine (5 mL) in methanol (25 mL) is added palladium on carbon (5%, 150 mg) under argon. The mixture was purged with a hydrogen flow and then stirred for two hours under a hydrogen atmosphere at atmospheric pressure. The reaction mixture is then filtered through a short pad of Celite, the filtrate was concentrated to afford 2.4 g (96%) of the desired compound.

The following scheme utilizes intermediate 16 for the preparation of other Category I analogs.

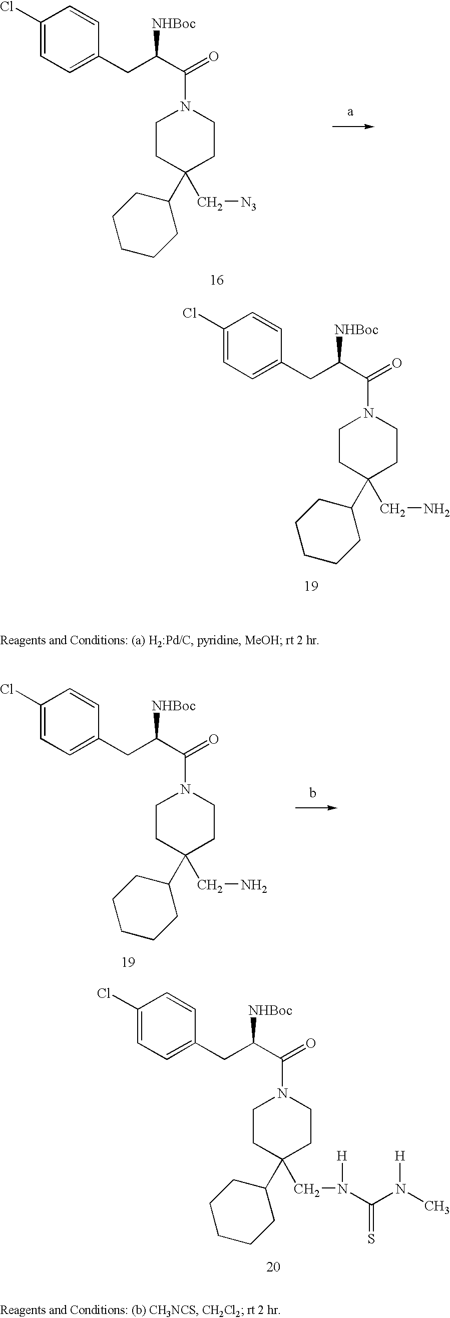

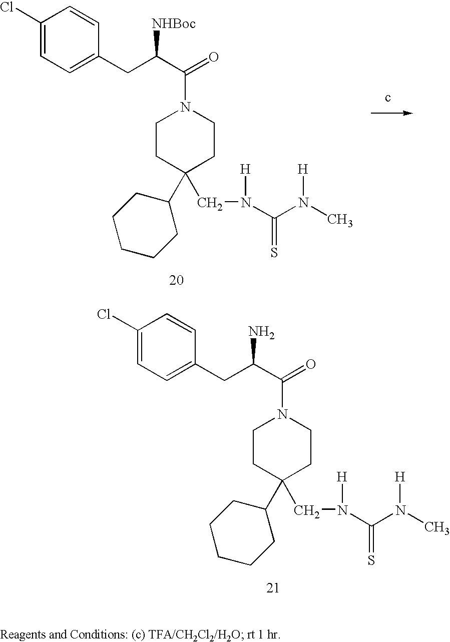

### EXAMPLE 3

### 1-{1-[2-Amino-3-(4-chlorophenyl)propionyl]-4-cyclohexyl-piperidin-4-ylmethyl}-3-methyl thiourea (21)

Preparation of [2-(4-aminoethyl-4-cyclohexyl-piperidin-1-yl)-1-R-(4-chloro-benzyl)-2-oxo-ethyl]-carbamic acid tert-butyl ester (19): To a solution of [2-(4-azidomethyl-4-cyclohexyl-piperidin-1-yl)-1-(R)-(4-chlorobenzyl)-2-oxo-ethyl]-carbamic acid tert-butyl ester, 16, (5.04 g, 10 mmole) and pyridine (10 mL) in methanol (50 mL) is added palladium on carbon (5%, 300 mg) under argon. The mixture was purged with a hydrogen flow and then stirred for two hours under a hydrogen atmosphere at atmospheric pressure. The reaction mixture is then filtered through a short pad of Celite, the filtrate was concentrated to afford 4.6 g (96%) of the desired compound.

Preparation of (1-(4-chlorobenzyl)-2-{4-cyclohexyl-4-[(3-methylthioureido)-methyl]piperidin-1-yl}-2-oxo-ethyl)-carbamic acid tert-butyl ester (20): To a stirred solution of [2-(4-aminoethyl-4-cyclohexyl-piperidin-1-yl)-1-R-(4-chloro-benzyl)-2-oxo-ethyl]-carbamic acid tert-butyl ester, 19, (46 mg, 0.096 mmol) in methylene chloride (6 mL) is added methyl isothiocyanate (10 mg, 0.11 mmol) and stirring continued for two hours at room temperature. The solvent is removed under reduced pressure and the residue washed with diethyl ether to afford the desired compound.

Preparation of 1-{1-[2-amino-3-(4-chlorophenyl)propionyl]-4-cyclohexyl-piperidin-4-ylmethyl}-3-methyl thiourea (21): A ready-to-use solution of trifluoroacetic acid:methylene chloride:water (1:1:0.1, 2 ml) is added to (1-(4-chlorobenzyl)-2-{4-cyclohexyl-4-[(3-methylthioureido)methyl]piperidin-1-yl}-2-oxo-ethyl)-carbamic acid tert-butyl ester, 20, (0.5 g, 1 mmol) and the reaction mixture is stirred for 0.5-1.0 hour. The mixture was concentrated under reduced pressure and partitioned between aqueous sodium bicarbonate and ethyl acetate. The organics were separated and the solvent removed under reduced pressure. The crude product is purified by preparative HPLC to give the title compound as the trifluoroacetic acid salt (100%).

The following scheme utilizes intermediate 6 for the preparation of other Category I analogs.

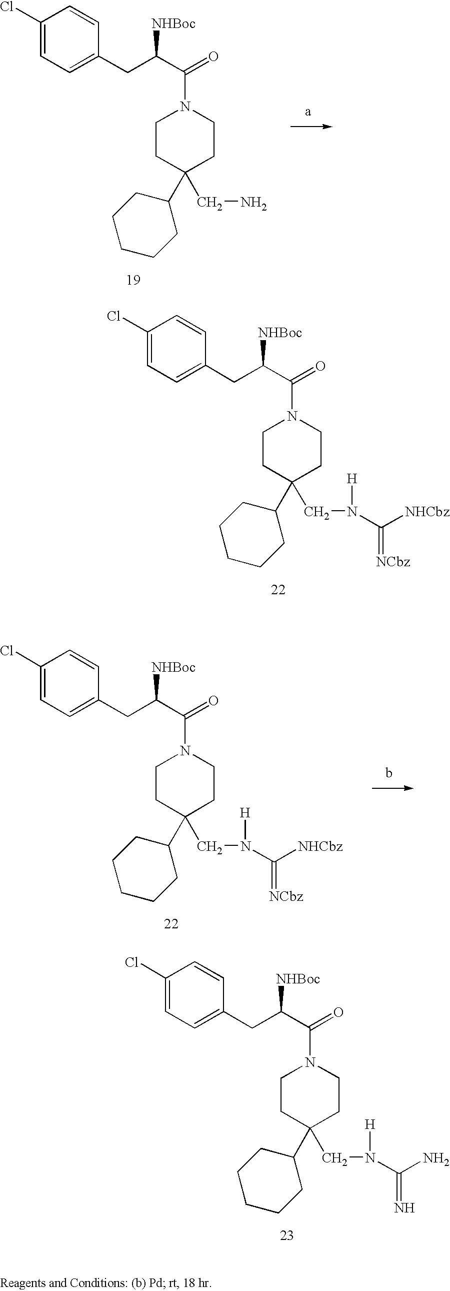

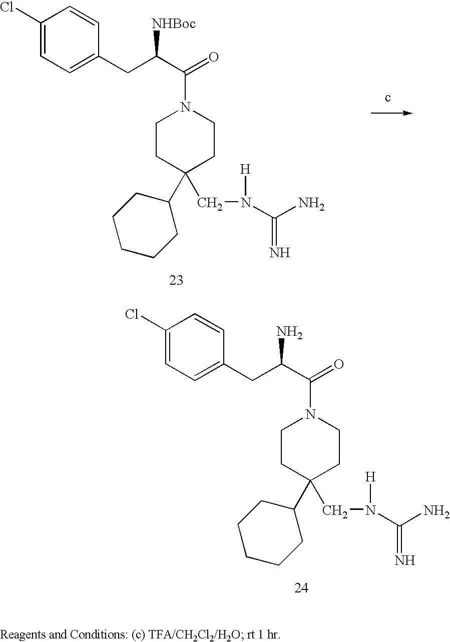

### EXAMPLE 4

### N-{1-[2-Amino-3-(4-chlorophenyl)propionyl]-4-cyclohexyl-piperidin-4-ylmethyl}-guanidine (24)

Preparation of {2-[4-cyclohexyl-4-(di-carbobenzyloxyguanidinyl)piperidin-1-yl]-1-(4-chlorobenzyl)-2-oxo-ethyl} carbamic acid tert-butyl ester (22): Mercury (II) chloride (401 mg, 0.48 mmol) is added to a stirred solution of [2-(4-aminoethyl-4-cyclohexyl-piperidin-1-yl)-1-R-(4-chloro-benzyl)-2-oxo-ethyl]-carbamic acid tert-butyl ester, 19, (588 mg, 1.23 mmol), 1,3-bis (benzoxycarbonyl)-2-methyl-thiopseudo urea (441 mg, 1.23 mmol) and triethylamine (0.62 mL, 5.64 mmol) in DMF (15 mL). The reaction mixture is stirred for 1 hour, diluted with EtOAc and filtered through a pad of Celite. The filtrate is concentrated in vacuo and the residue is purified over silica to afford the desired product.

Preparation of [1-(4-chlorobenzyl)-2-(4-cyclohexyl-4-gunidinomethyl-piperidin-1-yl)-2-oxo-ethyl]-carbamic acid tert-butyl ester (23): To a solution (100 mg) in {2-[4-cyclohexyl-4-(di-carbobenzyloxyguanidinyl)-piperidin-1-yl]-1-(4-chlorobenzyl)-2-oxo-ethyl} carbamic acid tert-butyl ester, 22, MeOH (3 mL) is added 10% Pd/C (12 g) under argon blanketing. The resulting slurry is purged with a hydrogen flow and then stirred for 2 hours under an atmosphere of hydrogen. The reaction mixture is then filtered through a short bed of Celite to remove the catalyst and the filtrate concentrated in vacuo to afford the desired product.

Preparation of N-{1-[2-Amino-3-(4-chlorophenyl)propionyl]-4-cyclohexyl-piperidin-4-ylmethyl}-guanidine (24): A solution of trifluoroacetic acid/dichloro-methane/water (1:1:0.1, 20 mL) is added to of [1-(4-chlorobenzyl)-2-(4-cyclohexyl-4-guanidinomethyl-piperidin-1-yl)-2-oxo-ethyl]-carbamic acid tert-butyl ester, 23, (5.24 g, 6.65 mmol) and the reaction mixture is allowed to stir for 30 to 60 minutes. The reaction solution is then concentrated in vacuo and partitioned between aqueous NaHCO₃ and EtOAc. The organic phase is concentrated in vacuo and the crude product purified via HPLC over silica gel to afford the desired product.

The following scheme utilizes intermediate 3 for the preparation of other Category I analogs.

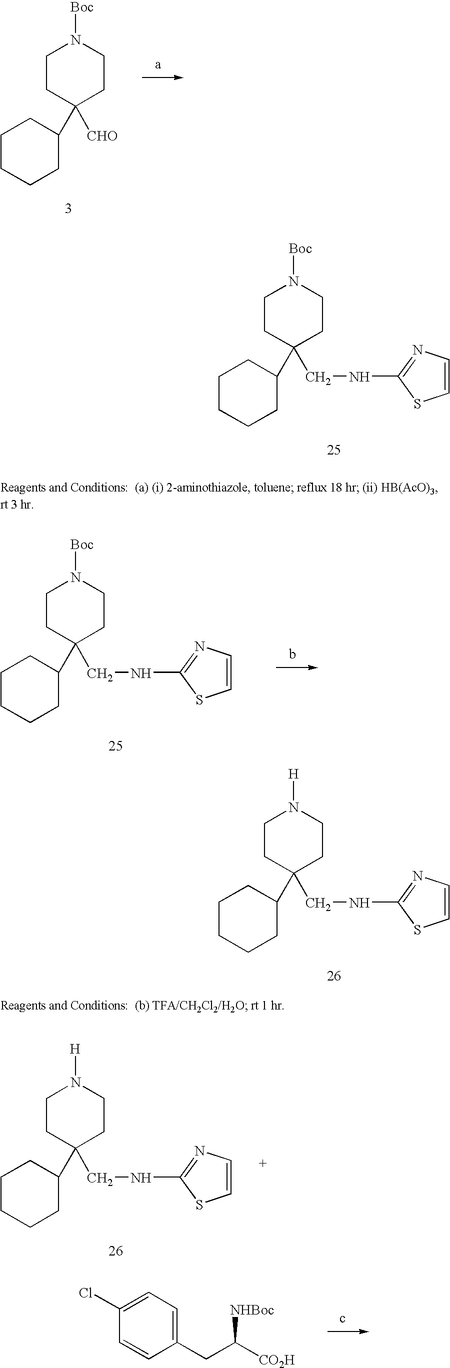

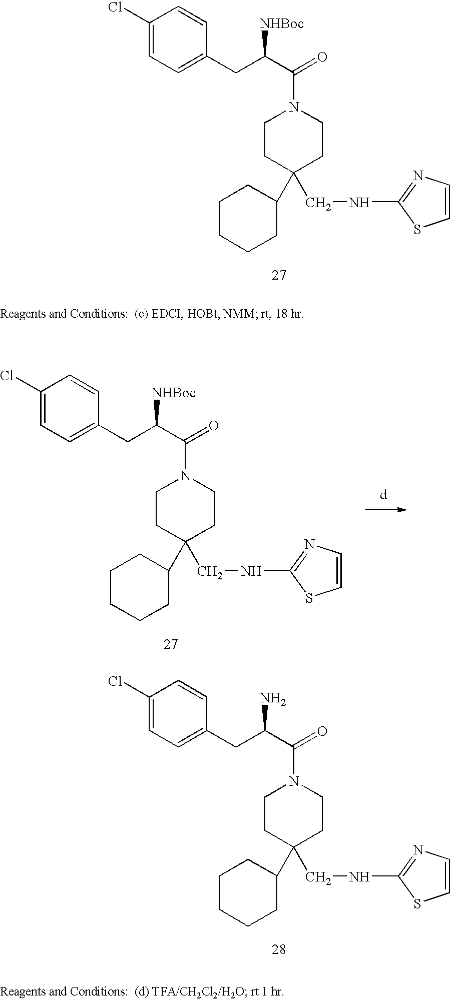

### EXAMPLE 5

### 2-R-Amino-3-(4-chloro-phenyl)-1-[4-cyclohexyl-4-(thiazol-2-ylaminomethyl)-piperidin-1-yl]-propan-1-one (28)

Preparation of 4-cyclohexyl-4-(thiazol-2-ylaminomethyl)-piperidine-1-carboxylic acid tert-butyl ester (25): 4-Cyclohexyl-4-formyl-piperidine-1-carboxylic acid tert-butyl ester, 3, (296 mg, 1.0 mmol) and 2-aminothiazole (103 mg, 1.0 mmol) are dissolved in toluene (15 mL), and the mixture was refluxed using a Dean-Stark apparatus overnight. The solution is then cooled to room temperature and sodium triacetoxyborohydride added. The reaction is stirred at room temperature for three hours and then diluted with ethyl acetate. The reaction mixture is washed with aqueous sodium bicarbonate and brine. The solvent is removed under reduced pressure and the residue purified by preparative HPLC to afford 312 mg (82% yield) of the desired compound. MS (ESI) m/z 380 (M+H⁺)

Preparation of (4-cyclohexyl-piperidin-4-ylmethyl)-thiazol-2-yl-amine (26): A ready-to-use solution of trifluoroacetic acid:methylene chloride:water (1:1:0.1, 7 mL) is added to 4-cyclohexyl-4-(thiazol-2-ylaminomethyl)-piperidine-1-carboxylic acid tert-butyl ester, 25, (312 mg, 0.82 mmol), and the reaction mixture is stirred for 0.5-1.0 hour. The mixture is then concentrated under reduced pressure and partitioned between aqueous sodium bicarbonate and ethyl acetate. The organics are separated and the solvent removed under reduced pressure. The crude product is purified by preparative HPLC to afford 220 mg (96% yield) of the desired compound as the trifluoroacetic acid salt.

Preparation of {1-(R)-(4-chlorobenzyl)-2-[4-cyclohexyl-4-(thiazol-2-ylaminomethyl)-piperidin-1-yl]-2-oxo-ethyl}-carbamic acid tert-butyl ester (27): To a solution of the (4-cyclohexyl-piperidin-4-ylmethyl)-thiazol-2-yl-amine, 26, (39 mg, 0.14 mmol), 2-(R)-tert-butoxycarbonylamino-3-(4-chloro-phenyl)-propionic acid (44 mg, 0.147 mmol), 1-hydroxybenzotriazole (38 mg, 0.28 mmol), 4-methylmorpholine (0.56 mmole, 62 □L) in N,N-dimethylformamide (7 mL) is added 1-(3-dimethylaminopropyl)-3-ethylcarbodiimide (35 mg, 0.183 mmol) and the reaction mixture stirred overnight. Aqueous ammonium chloride solution is then added and the reaction extracted with ethyl acetate. The organic layer is dried over sodium sulfate, filtered and concentrated under reduced pressure. The crude product is purified over silica to afford 48 mg (61% yield) of the desired compound. MS (ESI) m/z 561 (M+H⁺)

Preparation of 2-(R)-Amino-3-(4-chlorophenyl)-1-[4-cyclohexyl-4-(thiazol-2-ylaminomethyl)-piperidin-1-yl]-propan-1-one (28): A ready-to-use solution of trifluoroacetic acid:methylene chloride:water (1:1:0.1, 3 ml) is added to 1-R-(4-chlorobenzyl)-2-[4-cyclohexyl-4-(thiazol-2-ylaminomethyl)-piperidin-1-yl]-2-oxo-ethyl } -carbamic acid tert-butyl ester, 27, (48 mg, 0.086 mmole, and the reaction mixture stirred for 0.5-1.0 h. The mixture is then concentrated under reduced pressure and partitioned between, aqueous sodium bicarbonate and ethyl acetate. The solvent is removed under reduced pressure and the residue purified by preparative HPLC to afford 40 mg, (99% yield) of the desired compound as the trifluoroacetic acid salt. MS (ESI) m/z 461 (M+H⁺).

The compounds which comprise Category II of the melanocortin receptor ligands of the present invention are 4-cyclohexyl-4-[1,2,4]triazol-1-yl piperidines having the general scaffold:

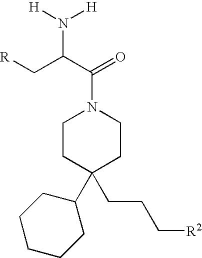

wherein R and R² are defined herein below in Table II.

TABLE II

| No. | R | R² |
|---|---|---|
| 49 | phenyl | —NH₂ |
| 50 | phenyl | imidazol-1-yl |
| 51 | phenyl | imidazol-2-yl |
| 52 | phenyl | imidazol-4-yl |
| 53 | phenyl | 1-methylimidazol-4-yl |
| 54 | phenyl | [1,2,4]triazol-1-yl |
| 55 | phenyl | —NHC(O)NHCH₃ |
| 56 | phenyl | —NHC(═NCN)NHCH₃ |
| 57 | phenyl | —NHC(═NCH₃)SCH₃ |
| 58 | phenyl | —NH(C═S)NHCH₃ |
| 59 | phenyl | (thiazol-2-yl)amino |
| 60 | phenyl | tetrazolyl |
| 61 | 4-fluorophenyl | —NH₂ |
| 62 | 4-fluorophenyl | imidazol-1-yl |
| 63 | 4-fluorophenyl | imidazol-2-yl |
| 64 | 4-fluorophenyl | imidazol-4-yl |
| 65 | 4-fluorophenyl | 1-methylimidazol-4-yl |
| 66 | 4-fluorophenyl | [1,2,4]triazol-1-yl |
| 67 | 4-fluorophenyl | —NHC(O)NHCH₃ |
| 68 | 4-fluorophenyl | —NHC(═NCN)NHCH₃ |
| 69 | 4-fluorophenyl | —NHC(═NCH₃)SCH₃ |
| 70 | 4-fluorophenyl | —NH(C═S)NHCH₃ |
| 71 | 4-fluorophenyl | (thiazole-2-yl)amino |
| 72 | 4-fluorophenyl | tetrazolyl |
| 73 | 4-chlorophenyl | —NH₂ |
| 74 | 4-chlorophenyl | imidazol-1-yl |
| 75 | 4-chlorophenyl | imidazol-2-yl |
| 76 | 4-chlorophenyl | imidazol-4-yl |
| 77 | 4-chlorophenyl | 1-methylimidazol-4-yl |
| 78 | 4-chlorophenyl | [1,2,4]triazol-1-yl |
| 79 | 4-chlorophenyl | —NHC(O)NHCH₃ |
| 80 | 4-chlorophenyl | —NHC(═NCN)NHCH₃ |
| 81 | 4-chlorophenyl | —NHC(═NCH₃)SCH₃ |
| 82 | 4-chlorophenyl | —NH(C═S)NHCH₃ |
| 83 | 4-chlorophenyl | (thiazole-2-yl)amino |
| 84 | 4-chlorophenyl | tetrazolyl |

The following is a scheme for preparing analogs encompassed by Category II of the melanocortin receptor ligands of the present invention.

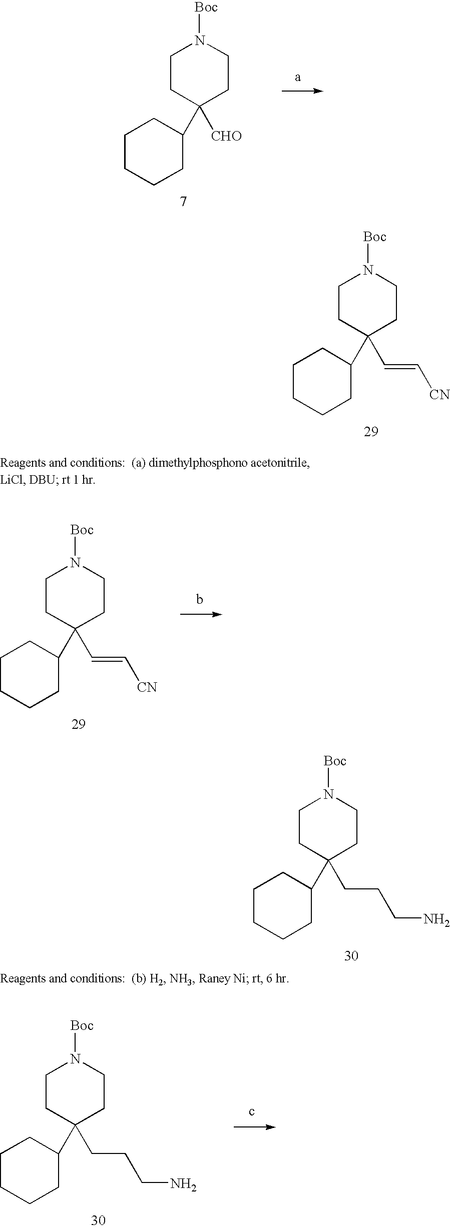

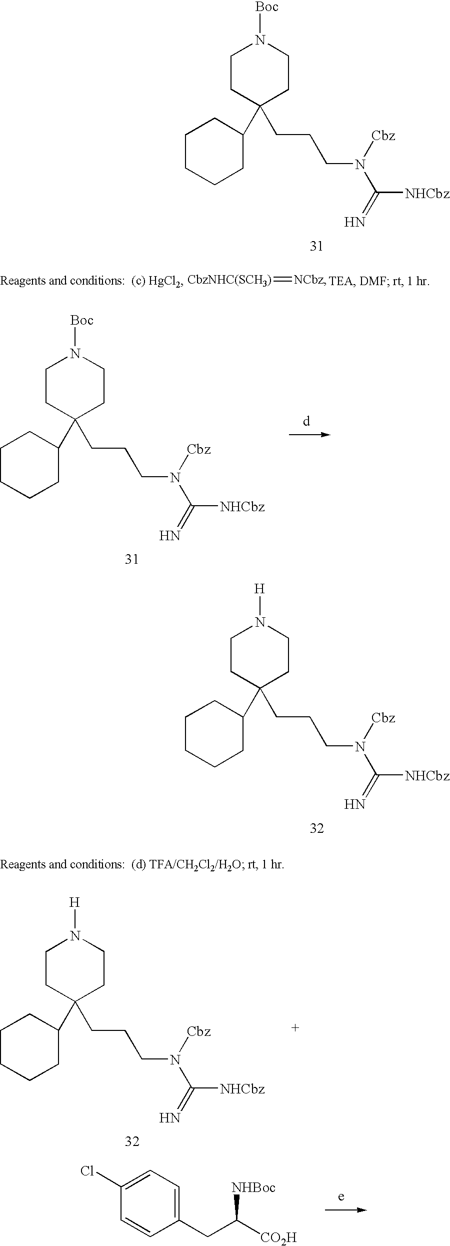

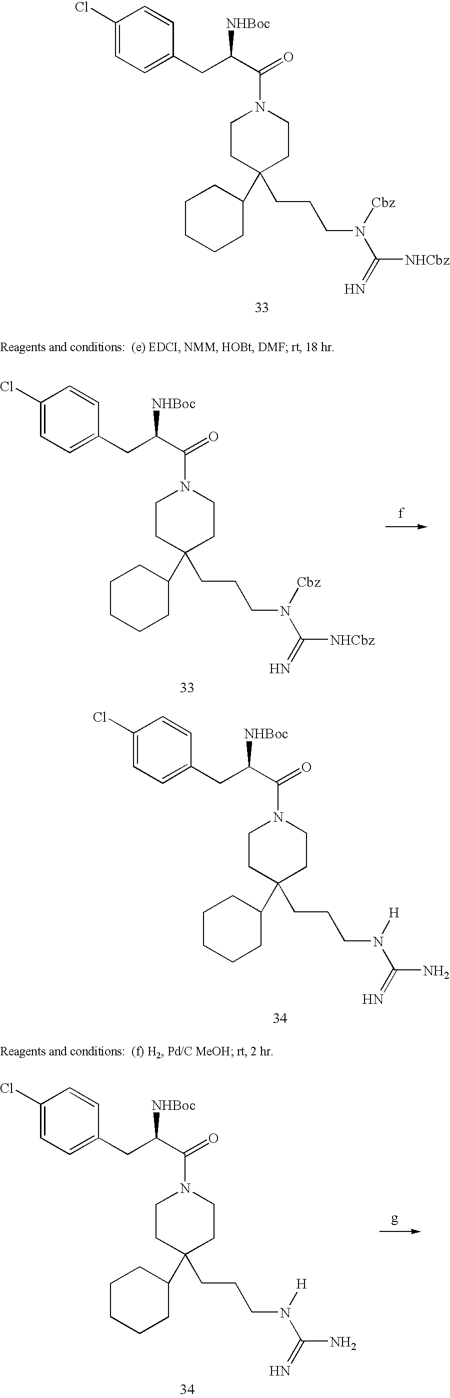

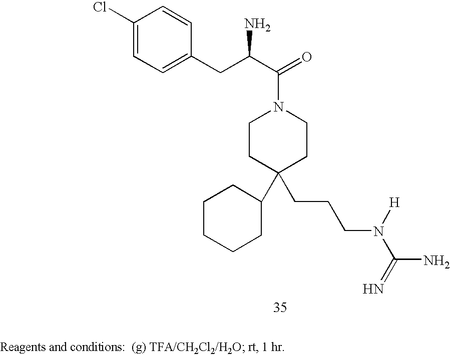

### EXAMPLE 6

### [2-[4-Cyclohexyl-4-(3-guanidino-propyl)-piperidin-1-yl]-1-R-(4-fluoro-benzyl)-2-oxo-ethyl]-carbamic acid tert-butyl ester (34):

Preparation of 4-(2-cyanovinyl)-4-cyclohexylpiperidine-1-carboxylic acid tert-butyl ester (29): To a solution of dimethyl phosphono acetonitrile (0.78 mL, 4.02 mmol), LiCl (184 mg, 4.02 mmol), and DBU (0.55 mL, 4.02 mmol) in anhydrous acetonitrile (25 mL) is added 4-cyclohexyl-4-formylpiperidine-1-carboxylic acid tert-butyl ester, 7, (992 mg, 3.35 mmol) under an atmosphere of argon at room temperature. The mixture is stirred for 1 hour and the solvent removed in vacuo. The resulting crude product is purified over silica gel eluting with dichloromethane/methanol 15:1 to afford the desired product in quantitative yield.

Preparation of 4-(3-aminopropyl)-4-cyclohexylpiperidine-1-carboxylic acid tert-butyl ester (30): To a solution of 4-(2-cyanovinyl)-4-cyclohexylpiperidine-1-carboxylic acid tert-butyl ester, 29, (800 mg, 2.35 mmol) in MeOH (33 mL) is added ammonia (16 mL) and Raney Ni (50 mg). The reaction mixture is degassed with nitrogen, purged with hydrogen gas and shaken under an atmosphere of hydrogen (45 psi) on a standard hydrogenation apparatus at room temperature for 6 hours. The reaction solution is filtered to remove the catalyst and the solvent removed in vacuo to afford the desired product was obtained as a colorless, sticky oil in quantitative yield.

Preparation of 4-cyclohexyl-4-(3-dicabobenzyloxy-guanidino-propyl)-piperidine-1-carboxylic acid tert-butyl ester (31): Mercury(II) chloride (401 mg, 0.48 mmol) is added to a stirred solution of 4-(3-aminopropyl)-4-cyclohexyl-piperidine-1-carboxylic acid tert-butyl ester, 30, (425 mg, 1.23 mmole), 1,3-bis(benzoxycarbonyl)-2-methyl-2-thiopseudo urea (441 mg, 1.23 mmol) and triethylamine (0.62 ml, 5.64 mmol) in N,N-dimethylformamide (15 ml). The reaction mixture is stirred for 1.0 hour and then diluted with ethyl acetate and filtered through a pad of Celite. The filtrate is concentrated under reduced pressure and the residue purified over silica (methylene chloride/acetone, 3:1) to afford 629 mg (78% yield) of the desired compound as a colorless solid.

Preparation of N-[3-(4-cyclohexyl-piperidin-4-yl)-propyl]-dicarbobenzyloxy-guanidine (32): A ready-to-use solution of trifluoroacetic acid:methylene chloride:water (1:1:0.1, 11 ml) is added to 4-cyclohexyl-4-(3-dicarbobenzyloxy-guanidino-propyl)-piperidine-1-carboxylic acid tert-butyl ester, 31, (300 mg, 0.46 mmole), and the reaction mixture is stirred for 0.5-1.0 hour. The mixture is then concentrated under reduced pressure and partitioned between aqueous sodium bicarbonate and ethyl acetate. The organics are separated and concentrated under reduced pressure. The crude product is purified by preparative HPLC to afford 254 mg (>99% yield) of the desired compound.

Preparation of [2-[4-cyclohexyl-4-(3-dicarbobenxyloxy-guanidino-propyl)-piperidin-1-yl]-1-R-(4-fluorobenzyl)-2-oxo-ethyl]-carbamic acid tert-butyl ester (33): To a solution of N-[3-(4-cyclohexyl-piperidin-4-yl)-propyl]-dicarbobenzyloxy-guanidine, 32, (36 mg, 0.055 mmol), 2-(R)-tert-butoxycarbonylamino-3-(4-fluorophenyl)-propionic acid (18.6 mg, 0.055 mmol), 1-hydroxybenzotriazole (14.9 mg, 0.11 mmol), 4-methylmorpholine (0.22 mmole, 24 ul) in N,N-dimethylformamide (3 ml) is added 1-(3-dimethylaminopropyl)-3-ethylcarbodiimide (14 mg, 0.07 mmol) and the reaction mixture stirred overnight. Aqueous ammonium chloride solution is then added and the reaction extracted with ethyl acetate. The organic layer is separated, dried over sodium sulfate, filtered and concentrated under reduced pressure. The crude product is purified over silica to afford 35 mg (77% yield) of the desired compound. MS (ESI) m/z 800, (M+H⁺).

Preparation of [2-[4-cyclohexyl-4-(3-guanidino-propyl)-piperidin-1-yl]-1-R-(4-fluoro-benzyl)-2-oxo-ethyl]-carbamic acid tert-butyl ester (34): To a solution of [2-[4-cyclohexyl-4-(3-dicarbobenzyloxy-guanidino-propyl)-piperidin-1-yl]-1-(R)-(4-fluoro-benzyl)-2-oxo-ethyl]-carbamic acid tert-butyl ester, 33, (100 mg) in methanol (3 mL) is added 10% palladium on carbon (12 mg) under argon. The mixture is purged with a hydrogen flow and then stirred for two hours under a hydrogen atmosphere at atmospheric pressure. The reaction mixture is then filtered through a short pad of Celite, and the filtrate concentrated under reduced pressure. The crude product is purified by preparative HPLC to afford 18 mg (98% yield) of the desired compound as the trifluoroacetic acid salt. MS (ESI) m/z 532, (M+H⁺).

Preparation of N-(3-{1-[2-Amino-3-(4-fluorophenyl)-propionyl]-4-cyclohexyl-piperidin-4-yl}-propyl)-guanidine (35): A ready-to-use solution of trifluoroacetic acid:methylene chloride:water (1:1:0.1, 11 mL) is added to [2-[4-cyclohexyl-4-(3-guanidino-propyl)-piperidin-1-yl]-1-R-(4-fluorobenzyl)-2-oxo-ethyl]-carbamic acid tert-butyl ester, 34, (35 mg, 0.042 mmol), and the reaction mixture is stirred for 0.5-1.0 hour. The mixture is concentrated under reduced pressure and partitioned between aqueous sodium bicarbonate and ethyl acetate. The organics are separated and concentrated under reduced pressure. MS (ESI) m/z 432, (M+H⁺).

The following are non-limiting examples of melanocortin receptor ligands according to the present invention.

2-Amino-3-(4-chlorophenyl)-1-(4-cyclohexyl-4-imidazol-1-ylmethyl-piperidin-1-yl)-propan-1-one;

2-Amino-3-(4-chlorophenyl)-1-(4-[1,2,4]triazol-1-ylmethyl-[4,4′]bipiperidin-1-yl)-propan-1-one;

2-Amino-3-(4-chlorophenyl)-1-(1′methanesulfonyl-4-[1,2,4]triazol-1-y-[4,4′]bipiperidin-1-yl)-propan-1-one;

2-Amino-3-(4-chlorophenyl)-1-[1′-methansulfonyl-4-(2-methyl-2H-tetrazol-5-ylmethyl-[4,4′]bipiperidinyl-1-yl]-propan 1-one;

2-Amino-3-(4-chlorophenyl)-1-[4-(2-methyl-2H-tetrazol-5-ylmethyl-[4,4′]bipiperidinyl-1-yl]-propan 1-one;

2-Amino-3-(4-chlorophenyl)-1-(4-cyclohexyl-4-pyrrol-1-ylmethyl-piperidin-1-yl)-propan-1-one;

2-Amino-3-(4-chlorophenyl)-1-[4-cyclohexyl-4-(1H-imidazol-4-ylmethyl)-piperidin-1-yl]-propan-1-one;

2-Amino-3-(4-chlorophenyl)-1-[4-cyclohexyl-4-(1-methyl-1H-imidazol-4-ylmethyl)-piperidin-1-yl]-propan-1-one;

-Amino-3-(4-chlorophenyl)-1-(4-cyclohexyl-4-thiophen-2-ylmethyl-piperidin-1-yl)-propan-1-one;

2-Amino-3-(4-chlorophenyl)-1-(4-cyclopentyl-4-imidazol-1-ylmethyl-piperidin-1-yl)-propan-1-one;

2-Amino-3-(4-chlorophenyl)-1-(4-cyclopentyl-4-pyrrol-1-ylmethyl-piperidin-1-yl)-propan-1-one;

2-Amino-3-(4-chlorophenyl)-1-[4-cyclopentyl-4-(1H-imidazol-4-ylmethyl)-piperidin-1-yl]-propan-1-one;

2-Amino-3-(4-chlorophenyl)-1-[4-cyclopentyl-4-(1-methyl-1H-imidazol-4-ylmethyl)-piperidin-1-yl]-propan-1-one;

2-Amino-3-(4-chlorophenyl)-1-(4-cyclopentyl-4-thiophen-2-ylmethyl-piperidin-1-yl)-propan-1-one;

2-Amino-3-(4-chlorophenyl)-1-(4-cyclopropyl-4-imidazol-1-ylmethyl-piperidin-1-yl)-propan-1-one;

2-Amino-3-(4-chlorophenyl)-1-(4-cyclopropyl-4-pyrrol-1-ylmethyl-piperidin-1-yl)-propan-1-one;

2-Amino-3-(4-chlorophenyl)-1-[4-cyclopropyl-4-(1H-imidazol-4-ylmethyl)-piperidin-1-yl]-propan-1-one;

2-Amino-3-(4-chlorophenyl)-1-[4-cyclopropyl-4-(1-methyl-1H-imidazol-4-ylmethyl)-piperidin-1-yl]-propan-1-one;

2-Amino-3-(4-chlorophenyl)-1-(4-cyclopropyl-4-thiophen-2-ylmethyl-piperidin-1-yl)-propan-1-one;

2-Amino-3-(4-chlorophenyl)-1-(4-cyclopropylmethyl-4-imidazol-1-ylmethyl-piperidin-1-yl)-propan-1-one;

2-Amino-3-(4-chlorophenyl)-1-(4-cycloheptyl-4-imidazol-1-ylmethyl-piperidin-1-yl)-propan-1-one;

2-Amino-3-(4-chlorophenyl)-1-(4′-imidazol-1-ylmethyl-[1,4′]bipiperidin-1′-yl)-propan-1-one;

2-Amino-3-(4-chlorophenyl)-1-(4-imidazol-1-ylmethyl-[4,4′]bipiperidin-1-yl)-propan-1-one;

2-Amino-3-(4-chlorophenyl)-1-(4-imidazol-1-ylmethyl-1′-methanesulfonyl-[4,4′]bipiperidin-1-yl)-propan-1-one;

2-Amino-3-(4-chlorophenyl)-1-(1′-acetyl-4-imidazol-1-ylmethyl-[4,4′]bipiperidin-1-yl)-propan-1-one;

2-Amino-3-(4-chlorophenyl)-1-[4′-(5H-[1,2,4]triazolyl-3-ylmethyl)-[1,4′]bipiperidin-1′-yl)-propan-1-one;

2-Amino-3-(4-chlorophenyl)-1-[4-(2-imidazol-1-yl-ethyl)-1′-methanesulfonyl-[4,4′]bipiperidin-1-yl)-propan-1-one;

1-[2-Amino-3-(4-chlorophenyl)-propionyl]-4-cyclohexylpiperidine-4-carboxylic acid [1,2,4]triazol-4-ylamide;

1-[2-Amino-3-(4-chlorophenyl)-propionyl]-4-cyclohexylpiperidine-4-carboxylic acid (2-methyl-3H-imidazol-4-yl)amide;

2-Amino-3-(4-chlorophenyl)-1-[4-cyclohexyl-4-(2-imidazol-1-ylethyl)-piperidin-1-yl]-propane-1-one;

2-Amino-3-(4-chlorophenyl)-1-[4-cyclopropyl-4-(2-imidazol-1-ylethyl)-piperidin-1-yl]-propane-1-one;

2-Amino-3-(4-chlorophenyl)-1-[4-cyclopropylmethyl-4-(2-imidazol-1-ylethyl)-piperidin-1-yl]-propane-1-one;

2-Amino-3-(4-chlorophenyl)-1-[4-thiophen-2-yl-4-(2-imidazol-1-ylethyl)-piperidin-1-yl]-propane-1-one;

2-Amino-3-(4-chlorophenyl)-1-[4-(2-methylene-cyclopentyl)methyl-4-(2-imidazol-1-ylethyl)-piperidin-1-yl]-propane-1-one;

2-{1-[2-Amino-3-(4-chlorophenyl)propionyl]-4-(2-imidazol-1-ylethyl)piperidin-4-ylmethyl}-cyclopentanone;

2-{1-[2-Amino-3-(4-chlorophenyl)propionyl]-4-imidazol-1-ylmethyl-piperidin-4-ylmethyl}-cyclopentanone;

2-{1-[2-Amino-3-(4-chlorophenyl)propionyl]-4-cyclohexylpiperidine-4-carboxylic acid (1H-[1,2,4]triazol-3-yl)amide;

2-{1-[2-Amino-3-(4-chlorophenyl)propionyl]-4-cyclohexylpiperidine-4-carboxylic acid (1-acetyl-1H-[1,2,4]triazol-3-yl)amide;

2-{1-[2-Amino-3-(4-chlorophenyl)propionyl]-4-cyclohexylpiperidine-4-carboxylic acid (1-methanesulfonyl-1H-[1,2,4]triazol-3-yl)amide;

### FORMULATIONS

The present invention also relates to compositions or formulations which comprise the melanocortin receptor ligands according to the present invention. In general, the compositions of the present invention comprise:

a) an effective amount of one or more melanocortin receptor ligands according to the present invention; and

b) one or more pharmaceutically acceptable excipients.

The compositions of this invention are typically provided in unit dosage form. For the purposes of the present invention the term “unit dosage form” is defined herein as comprising an effective amount of one or more melanocortin receptor ligands. The compositions of the present invention contain in one embodiment from about 1 mg to about 750 mg of one or more melanocortin receptor ligands, while in other embodiments the compositions comprise from about 3 mg to about 500 mg, or from about 5 mg to about 300 mg respectively.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

Non-limiting examples of substances which can serve as pharmaceutically-acceptable excipients or components thereof are sugars, inter alia, lactose, glucose and sucrose, sorbitol, mannitol; starches, inter alia, corn starch and potato starch; cellulose and its derivatives, inter alia, sodium carboxymethyl cellulose, ethyl cellulose, and methyl cellulose; powdered tragacanth; malt; gelatin; talc; solid lubricants, such as stearic acid and magnesium stearate; vegetable oils, propylene glycol, glycerin, and polyethylene glycol; agar; alginic acid; wetting agents and lubricants, inter alia, sodium lauryl sulfate; coloring agents; flavoring agents; tableting agents, stabilizers; antioxidants; preservatives; pyrogen-free water; isotonic saline; and buffers.

Standard pharmaceutical formulation techniques are disclosed in Remington's Pharmaceutical Sciences, Mack Publishing Company, Easton, Pa., latest edition and Peptide and Protein Drug Delivery, Marcel Dekker, NY, 1991. Dosage forms useful for making the compositions of the present invention or which are compatible with the methods of use as described herein below are described in the following references, all incorporated by reference herein: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

The present invention further relates to forms of the present compounds, which under normal human or higher mammalian physiological conditions, release the compounds described herein. One iteration of this aspect includes the pharmaceutically acceptable salts of the analogs described herein. The formulator, for the purposes of compatibility with delivery mode, excipients, and the like, can select one salt form of the present analogs over another since the compounds themselves are the active species which mitigate the disease processes described herein.

Related to this aspect are the various precursor or “pro-drug” forms of the analogs of the present invention. It may be desirable to formulate the compounds of the present invention as a chemical species which itself is not a melanocortin receptor ligand as described herein, but instead are forms of the present analogs which when delivered to the body of a human or higher mammal will undergo a chemical reaction catalyzed by the normal function of the body, inter alia, enzymes present in the stomach, blood serum, said chemical reaction releasing the parent analog. Or alternatively, said “pro-drug” form may cross the blood/brain barrier before undergoing a change which releases the melanocortin receptor ligand in its active form. The term “pro-drug” relates to these species which are converted in vivo to the active pharmaceutical.

### METHOD OF USE

The present invention also relates to a method for controlling one or more melanocortin receptor, MC-3 or MC-4, mediated or melanocortin receptor modulated mammalian diseases or conditions, said method comprising the step of administering to a human or higher mammal an effective amount of a composition comprising one or more of the melanocortin receptor ligands according to the present invention.

Because the melanocortin receptor ligands of the present invention can be delivered in a manner wherein more than one site of control can be achieved, more than one disease state can be modulated at the same time. Non-limiting examples of diseases which are affected by an antagonist or agonist which stimulates the MC-3 or MC-4 receptor, obesity and other body weight disorders, inter alia, anorexia and cachexia. Utilizing the melanocortin receptor ligands of the present invention will therefore affect a variety of diseases, disease states, conditions, or syndromes resulting from body weight disorders, inter alia, insulin resistance, glucose intolerance, Type-2 diabetes mellitus, coronary artery disease, elevated blood pressure, hypertension, dyslipidaemia, cancer (e.g., endometrial, cervical, ovarian, breast, prostate, gallbladder, colon), menstrual irregularities, hirsutism, infertility, gallbladder disease, restrictive lung disease, sleep apnea, gout, osteoarthritis, and thromboembolic disease.

MC-3 and MC-4 receptor ligands are also effective in treating disorders relating to behavior, memory (including learning), cardiovascular function, inflammation, sepsis, cardiogenic and hypovolemic shock, sexual dysfunction, penile erection, muscle atrophy, nerve growth and repair, intrauterine fetal growth, and the like.

Although the melanocortin receptor ligands of the present invention are discrete chemical entities, the method of delivery or the method of use may be coupled with other suitable drug delivery systems. For example, a drug delivery technique useful for the compounds of the present invention is the conjugation of the compound to an active molecule capable of being transported through a biological barrier (see e.g. Zlokovic, B. V., Pharmaceutical Research, Vol. 12, pp. 1395-1406 (1995)). A specific example constitutes the coupling of the compound of the invention to fragments of insulin to achieve transport across the blood brain barrier (Fukuta, M., et al. Pharmaceutical Res., Vol. 11, pp. 1681-1688 (1994)). For general reviews of technologies for drug delivery suitable for the compounds of the invention see Zlokovic, B. V., Pharmaceutical Res., Vol. 12, pp. 1395-1406 (1995) and Pardridge, WM, Pharmacol. Toxicol., Vol. 71, pp. 3-10 (1992).

### PROCEDURES

The compounds of the present invention can be evaluated for efficacy, for example, measurements of cytokine inhibition constants, Kᵢ, and IC₅₀ values can be obtained by any method chosen by the formulator.

Non-limiting examples of suitable assays include:

i) UV-visible substrate enzyme assay as described by L. Al Reiter, Int. J. Peptide Protein Res., 43, 87-96 (1994).

ii) Fluorescent substrate enzyme assay as described by Thornberry et al., Nature, 356, 768-774 (1992).

iii) PBMC Cell assay as described in U.S. Pat. No. 6,204,261 B1 Batchelor et al., issued Mar. 20, 2001.

iv) accumulation of second messenger elements such as cAMP described by Chen et al., Anal Biochem. 226, 349-54, (1995).

Each of the above citations is included herein by reference.

Functional activity (in vitro pre-screening) can be evaluated using various methods known in the art. For example, measurement of the second messenger, cAMP, as described in citation (iv) above, evaluation by Cytosensor Microphysiometer techniques (Boyfield et al. 1996), or by using the compounds of the invention alone, or in combination with natural or synthetic MSH-peptides.

The compounds of the present invention will interact preferentially (i.e., selectively) to MC-4 and/or MC-3, relative to the other melanocortin receptors. Selectivity is particularly important when the compounds are administered to humans or other animals, to minimize the number of side effects associated with their administration. MC-3/MC-4 selectivity of a compound is defined herein as the ratio of the EC₅₀ of the compound for an MC-1 receptor (“EC₅₀-MC-1”) over the EC₅₀ of the compound for the MC-3 (EC₅₀-MC-3)/MC-4 (EC₅₀-MC-4) receptor, the EC₅₀ values being measured as described above. The formulas are as follows:

MC-3 selectivity=[EC₅₀-MC-1]/[EC₅₀-MC-3]

MC-4 selectivity=[EC₅₀-MC-1]/[EC₅₀-MC-4]

For the purposes of the present invention a receptor ligand (analog) is defined herein as being “selective for the MC-3 receptor” when the above-mentioned ratio “MC-3-selectivity” is at least about 10. In other treatments, methods, or compositions this value is at least about 100, while for yet other embodiments of the present invention the selectivity is at least about 500. A compound is defined herein as being “selective for the MC-4 receptor” when the above-mentioned ratio “MC-3-selectivity” is at least about 10. In other treatments, methods, or compositions this value is at least about 100, while for yet other embodiments of the present invention the selectivity is at least about 500.

While particular aspects of the present invention and embodiments thereof have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound, including all enatiomeric and diasteriomeric forms and pharmaceutically acceptable salts thereof, said compound having the formula: 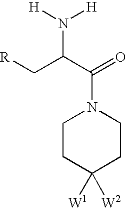
wherein R is a substituted or unsubstituted hydrocarbyl unit selected from the group consisting of:
a) non-aromatic carbocyclic rings;
b) aromatic carbocyclic rings;
c) non-aromatic heterocyclic rings;
d) aromatic heterocyclic rings;
W¹ is a pendant unit having the formula:: 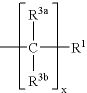
R¹ is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings; and
v) C₃-C₁₃ aromatic heterocyclic rings;
R^{3a} and R^{3b} are each independently selected from the group consisting of
i) hydrogen;
ii) methyl; and
iii) R^{3a} and R^{3b} can be taken together to form a carbonyl unit;
the index x has the value from 0 to 10;
W² is a pendant unit having the formula: 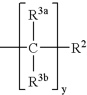
R² is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings;
v) C₃-C₁₃ aromatic heterocyclic rings;
vi) —C(Y)R⁴;
vii) —C(Y)₂R⁴;
viii) —C(Y)N(R⁴)₂;
ix) —C(Y)NR⁴N(R⁴)₂;
x) —CN;
xi) —[C(R⁴)₂]C(R⁴)₂;
xii) —N(R⁴)₂;
xiii) —NR⁴CN;
xiv) —NR⁵C(Y)R⁴;
xv) —NR⁵C(Y)N(R⁴)₂;
xvi) —NHN(R⁴)₂;
xvii) —NHOR⁴;
xviii) —NO₂;
xix) —OR⁴;
xx) and mixtures thereof;
Y is —O—, —S—, ═O, ═S, ═NR⁴, —R⁴, and mixtures thereof; R⁴ is hydrogen, C₁-C₄alkyl, —OH, and mixtures thereof; R⁵ is hydrogen, halogen, and mixtures thereof; M is hydrogen or a salt forming cation;
R^{3a} and R^{3b} are the same as above;
the index y has the value from 0 to 10.

2. A compound according to claim 1 wherein R units are selected from the group consisting of phenyl, 3-fluorophenyl, 4-fluorophenyl, 3,5-difluorophenyl, 4-chlorophenyl, 4-hydroxyphenyl, 4-methylphenyl, and 4-acetoxyphenyl.

3. A compound according to claim 1 wherein W¹ has the formula:
—R¹
and R¹ is selected from the group consisting of cyclohexyl, cyclopropyl, cyclopropylmethyl, cyclopentyl, and cycloheptyl.

4. A compound according to claim 3 wherein R¹ is cyclohexyl.

5. A compound according to claim 1 wherein W¹ has the formula:
—R¹
R¹ is selected from the group consisting of piperidin-1-yl, piperidin-4-yl, 1-methanesulfonylpiperidin-4-yl, 1-acetylpiperidin-4-yl, 2-cyclopentanone, cyclopentanon-2-ylmethyl, 2-methylenecyclopentylmethyl, and thiophen-2-yl.

6. A compound according to claim 1 wherein W² is a short chain substituted or non-substituted amide selected form the group consisting of —C(O)NHCH₃; —C(O)NHCH₂CH₃; —C(O)NHCH(CH₃)₂; —C(O)NHCH₂CH₂CH₃; —C(O)NH₂; —C(O)NHCH₂CH₂CH₂CH₃; —C(O)NHCH₂CH(CH₃)₂; —C(O)NHCH₂CH═CHCH₃; —C(O)NHCH₂CH₂CH(CH₃)₂; —C(O)NHCH₂C(CH₃)₃; —C(O)NHCH₂CH₂SCH₃; —C(O)NHCH₂CH₂OH; —NHC(O)CH₃; —NHC(O)CH₂CH₃; and —NHC(O)CH₂CH₂CH₃.

7. A compound according to claim 1 wherein W² unit has the formula:
(CH₂)_{y}—R²
the index y is 1, 2, or 3 and R² is selected from the group consisting of:
i) thiazolyl, 2-methylthiazolyl, 4-mentylthiazolyl, 5-methylthiazolyl having the formula: 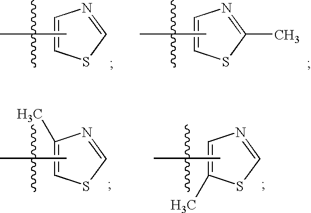
ii) 1,3,4-thiadiazolyl, 2-methyl-1,3,4-thiadiazolyl having the formula: 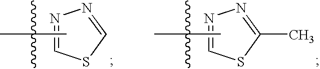
iii) 1,2,5-thiadiazolyl, 3-methyl-1,2,5-thiadiazolyl having the formula: 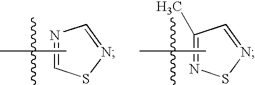
iv) oxazolyl, 2-methyloxazolyl, 4-methyloxazolyl, 5-methyloxazolyl having the formula: 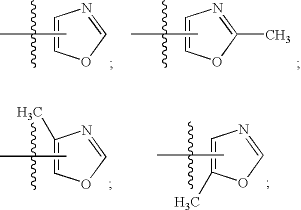
v) imidazolyl, 2-methylimidazolyl, 5-methylimidazolyl having the formula: 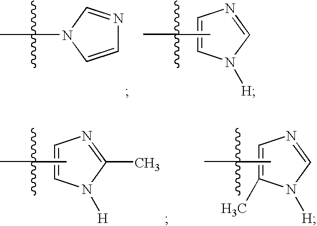
vi) 5-methyl-1,2,4-oxadiazolyl, 2-methyl-1,3,4-oxadiazolyl, 5-amino-1,2,4-oxadiazolyl, having the formula: 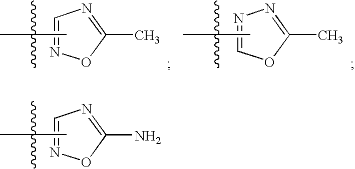
vii) 1,2-dihydro[1,2,4]triazol-3-one-1-yl, 2-methyl-1,2-dihydro[1,2,4]triazol-3-one-5-yl, having the formula: 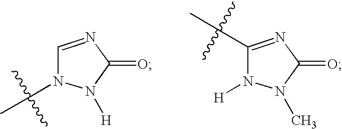
viii) oxazolidin-2-one-3-yl; 4,4-dimethyloxazolidin-2-one-3-yl; imidazolidin-2-one-1-yl; 1-methylimidazolidin-2-one-1-yl, having the formula: 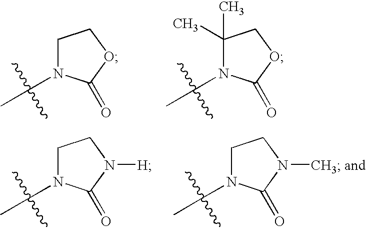
ix) 2-methyl-1,3,4-oxadiazolyl, 2-amino-1,3,4-oxadiazolyl, 2-(N,N-dimethylamino)-1,3,4-oxadiazolyl, having the formula:

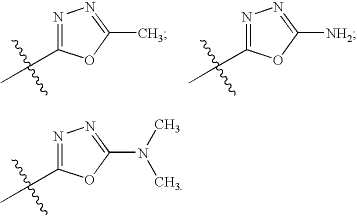

8. A compound according to claim 1 wherein W² unit has the formula:
—(CH₂)ₓ—R²
the index x is 1, 2, or 3 and R² is selected from the group consisting of:
i) triazoles having the formula: 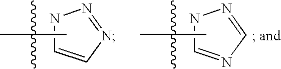
ii) tetrazole having the formula:

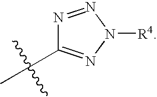

9. A compound according to claim 1 having the formula: 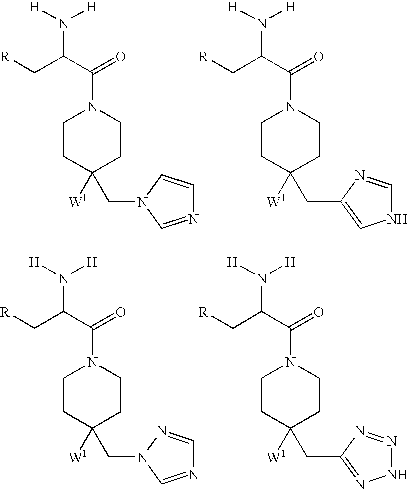
wherein R is selected from the group consisting of phenyl, 3-fluorophenyl, 4-fluorophenyl, 3,5-difluorophenyl, 4-chlorophenyl, 4-hydroxyphenyl, 4-methylphenyl, and 4-acetoxy-phenyl.

10. A compound according to claim 9 wherein R is 3-chlorophenyl.

11. A compound according to claim 9 wherein W¹ is has the formula:
—R¹
wherein R¹ units which are substituted and unsubstituted carbocyclic rings selected from the group consisting of cyclopropyl, cyclopentyl, cyclohexyl, 2-methylenecyclopentyl, and cycloheptyl.

12. A compound according to claim 1 wherein W¹ has the formula:
—R¹
R¹ is selected from the group consisting of piperidin-1-yl, piperidin-4-yl, 1-methanesulfonylpiperidin-4-yl, 1-acetylpiperidin-4-yl, 2-cyclopentanone, cyclopentanon-2-ylmethyl, 2-methylenecyclopentylmethyl, and thiophen-2-yl.

13. A compound according to claim 1 wherein W² unit has the formula:
—(CH₂)_{y}R²
the index y is 1, 2, or 3 and R² is selected from the group consisting of:
a) —C(O)N(R⁴)₂;
b) —C(O)NR⁴N(R⁴)₂;
c) —NR⁴C(O)N(R⁴)₂; and
d) —NR⁴C(═NR⁴)N(R⁴)₂
R⁴ is hydrogen, methyl, —NO₂, —CN, and mixtures thereof.

14. A compound according to claim 13 wherein W² has the formula:
a) —(CH₂)_{y}NHC(O)NH₂;
b) —(CH₂)_{y}NHC(═NH)NH₂;
c) —(CH₂)_{y}NHC(═NCH₃)NHCN;
d) —(CH₂)_{y}NHC(═NNO₂)NHCN;
e) —(CH₂)_{y}NHC(═NCH₃)NHNO₂;
f) —(CH₂)_{y}NHC(═NCN)NHNO₂; and
g) —(CH₂)_{y}NHC(═NCN)NH₂;
wherein y is 1, 2, or 3.

15. A compound according to claim 14 having the formula: 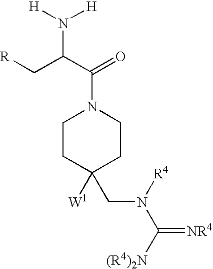
wherein R⁴ is hydrogen, methyl, —CN, —NO₂, and mixtures thereof.

16. A compound according to claim 15 wherein R is selected from the group consisting of phenyl, 3-fluorophenyl, 4-fluorophenyl, 3,5-difluorophenyl, 4-chlorophenyl, 4-hydroxyphenyl, 4-methylphenyl, and 4-acetoxy-phenyl.

17. A compound according to claim 16 wherein R is 3-chlorophenyl.

18. A compound, or a pharmaceutically acceptable salt thereof, having the formula: 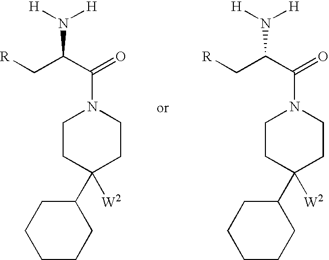
wherein R is a substituted or unsubstituted aromatic carbocyclic ring;
W² is a pendant unit having the formula:
—(CH₂)_{y}R²
R² is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings;
v) C₃-C₁₃ aromatic heterocyclic rings;
vi) —C(Y)R⁴;
vii) —C(Y)₂R ;
viii) —C(Y)N(R⁴)₂;
ix) —C(Y)NR⁴N(R⁴)₂;
x) —CN;
xi) —[C(R⁴)₂]C(R⁴)₂;
xii) —N(R⁴)₂;
xiii) —NR⁴CN;
xiv) —NR⁵C(Y)R⁴;
xv) —NR⁵C(Y)N(R⁴)₂;
xvi) —NHN(R⁴)₂;
xvii) —NHOR⁴;
xviii) —NO₂;
xix) —OR⁴;
xx) and mixtures thereof;
Y is —O—, —S—, ═O, ═S, ═NR⁴, —R⁴, and mixtures thereof; R⁴ is hydrogen, C₁-C₄ linear, branched, or cyclic alkyl, —OH, —CN, —NO₂, and mixtures thereof; R⁵ is hydrogen, halogen, and mixtures thereof; M is hydrogen or a salt forming cation; y is an index having the value of 1, 2, or 3.

19. A compound according to claim 18 wherein R units are selected from the group consisting of phenyl, 3-fluorophenyl, 4-fluorophenyl, 3,5-difluorophenyl, 4-chlorophenyl, 4-hydroxyphenyl, 4-methylphenyl, and 4-acetoxyphenyl.

20. A compound according to claim 18 wherein R² is selected from the group consisting of:
i) thiazolyl, 2-methylthiazolyl, 4-mentylthiazolyl, 5-methylthiazolyl having the formula: 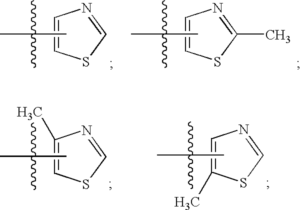
ii) 1,3,4-thiadiazolyl, 2-methyl-1,3,4-thiadiazolyl having the formula: 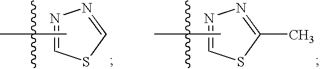
iii) 1,2,5-thiadiazolyl, 3-methyl-1,2,5-thiadiazolyl having the formula: 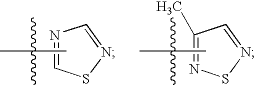
iv) oxazolyl, 2-methyloxazolyl, 4-methyloxazolyl, 5-methyloxazolyl having the formula: 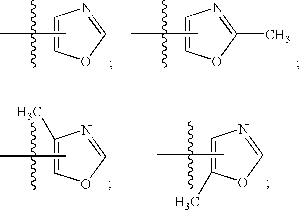
v) imidazolyl, 2-methylimidazolyl, 5-methylimidazolyl having the formula: 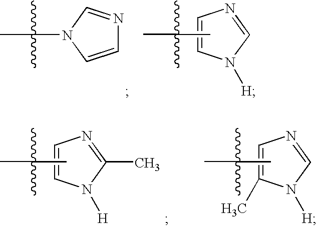
vi) 5-methyl-1,2,4-oxadiazolyl, 2-methyl-1,3,4-oxadiazolyl, 5-amino-1,2,4-oxadiazolyl, having the formula: 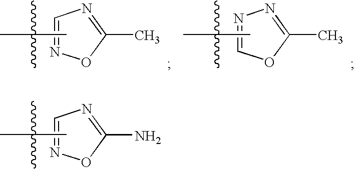
vii) 1,2-dihydro[1,2,4]triazol-3-one-1-yl, 2-methyl-1,2-dihydro[1,2,4]triazol-3-one-5-yl, having the formula: 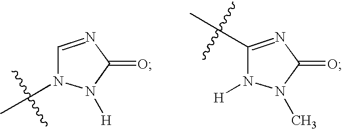
viii) oxazolidin-2-one-3-yl; 4,4-dimethyloxazolidin-2-one-3-yl; imidazolidin-2-one-1-yl; 1-methylimidazolidin-2-one-1-yl, having the formula: 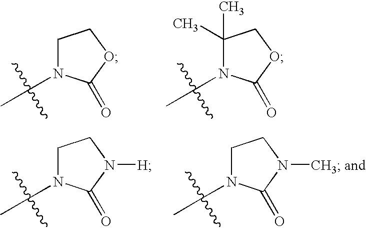
ix) 2-methyl-1,3,4-oxadiazolyl, 2-amino-1,3,4-oxadiazolyl, 2-(N,N-dimethylamino)-1,3,4-oxadiazolyl, having the formula:

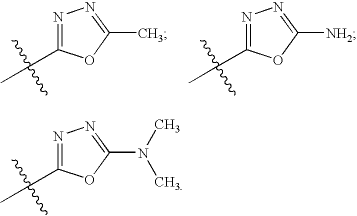

21. A compound according to claim 18 selected from the group having the formula:

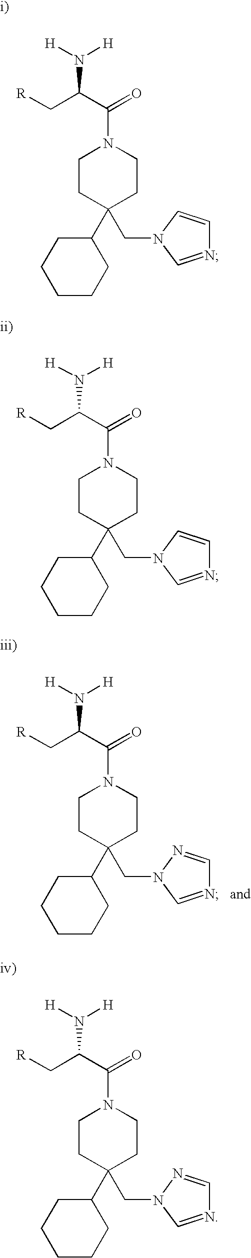

22. A compound according to claim 21 wherein R is selected from the group consisting of phenyl, 3-fluorophenyl, 4-fluorophenyl, 3,5-difluorophenyl, 4-chlorophenyl, 4-hydroxyphenyl, 4-methylphenyl, and 4-acetoxyphenyl.

23. A composition comprising:
A) an effective amount of one or more melanocortin receptor ligands, said ligands having all enatiomeric and diasteriomeric forms and their pharmaceutically acceptable salts, said ligands having the formula: 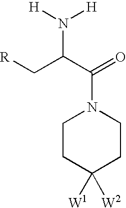
wherein R is a substituted or unsubstituted hydrocarbyl unit selected from the group consisting of:
a) non-aromatic carbocyclic rings;
b) aromatic carbocyclic rings;
c) non-aromatic heterocyclic rings;
d) aromatic heterocyclic rings;
W¹ is a pendant unit having the formula:: 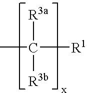
R¹ is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings; and
v) C₃-C₁₃ aromatic heterocyclic rings;
R^{3a} and R^{3b} are each independently selected from the group consisting of
i) hydrogen;
ii) methyl; and
iii) R^{3a} and R^{3b} can be taken together to form a carbonyl unit;
the index x has the value from 0 to 10;
W² is a pendant unit having the formula: 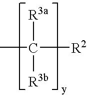
R² is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings;
v) C₃-C₁₃ aromatic heterocyclic rings;
vi) —C(Y)R⁴;
vii) —C(Y)₂R;
viii) —C(Y)N(R⁴)₂;
ix) —C(Y)NR⁴N(R⁴)₂;
x) —CN;
xi) —[C(R⁴)₂]C(R⁴)₂;
xii) —N(R⁴)₂;
xiii) —NR⁴CN;
xiv) —NR⁵C(Y)R⁴;
xv) —NR⁵C(Y)N(R⁴)₂;
xvi) —NHN(R⁴)₂;
xvii) —NHOR⁴;
xviii) —NO₂;
xix) —OR⁴;
xx) and mixtures thereof;
Y is —O—, —S—, ═O, ═S, ═NR⁴, —R⁴, and mixtures thereof; R⁴ is hydrogen, C₁-C₄alkyl, —OH, and mixtures thereof; R⁵ is hydrogen, halogen, and mixtures thereof; M is hydrogen or a salt forming cation;
R^{3a} and R^{3b} are the same as above;
the index y has the value from 0 to 10; and
B) one or more pharmaceutically acceptable excipients.

24. A method for controlling weight gain in a human or higher mammal, said method comprising the step of administering to said human or higher mammal an effective amount of one or more melanocortin receptor ligands, said ligands having all enatiomeric and diasteriomeric forms and their pharmaceutically acceptable salts, said ligands having the formula: 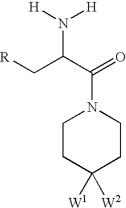
wherein R is a substituted or unsubstituted hydrocarbyl unit selected from the group consisting of:
a) non-aromatic carbocyclic rings;
b) aromatic carbocyclic rings;
c) non-aromatic heterocyclic rings;
d) aromatic heterocyclic rings;
W¹ is a pendant unit having the formula:: 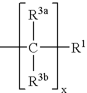
R¹ is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings; and
v) C₃-C₁₃ aromatic heterocyclic rings;
R^{3a} and R^{3b} are each independently selected from the group consisting of
i) hydrogen;
ii) methyl; and
iii) R^{3a} and R^{3b} can be taken together to form a carbonyl unit;
the index x has the value from 0 to 10;
W² is a pendant unit having the formula: 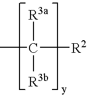
R² is selected from the group consisting of:
i) hydrogen;
ii) C₃-C₈ non-aromatic carbocyclic rings;
iii) C₆-C₁₄ aromatic carbocyclic rings;
iv) C₁-C₇ non-aromatic heterocyclic rings;
v) C₃-C₁₃ aromatic heterocyclic rings;
vi) —C(Y)R⁴;
vii) —C(Y)₂R;
viii) —C(Y)N(R⁴)₂;
ix) —C(Y)NR⁴N(R⁴)₂;
x) —CN;
xi) —[C(R⁴)₂]C(R⁴)₂;
xii) —N(R⁴)₂;
xiii) —NR⁴CN;
xiv) —NR⁵C(Y)R⁴;
xv) —NR⁵C(Y)N(R⁴)₂;
xvi) —NHN(R⁴)₂;
xvii) —NHOR⁴;
xviii) —NO₂;
xix) —OR⁴;
xx) and mixtures thereof;
Y is —O—, —S—, ═O, ═S, ═NR⁴, —R⁴, and mixtures thereof; R⁴is hydrogen, C₁-C₄alkyl, —OH, and mixtures thereof; R⁵ is hydrogen, halogen, and mixtures thereof; M is hydrogen or a salt forming cation;
R^{3a} and R^{3b} are the same as above;
the index y has the value from 0 to 10.
